(19) United States
(12) Patent Application Publication
(10) Pub. No.: US 2025/0241583 A1
(43) Pub. Date: Jul. 31, 2025

(54) TOUCH-BASED BIOMARKER MONITORING SYSTEM

(71) Applicant: Persperion Diagnostics Inc., San Diego, CA (US)

(72) Inventors: Lu YIN, San Diego, CA (US); Jongmin Moon, San Diego, CA (US); Saeromi Chung, San Diego, CA (US)

(21) Appl. No.: 19/078,191

(22) Filed: Mar. 12, 2025

Related U.S. Application Data
(63) Continuation of application No. PCT/US2024/036816, filed on Jul. 3, 2024.
(60) Provisional application No. 63/512,248, filed on Jul. 6, 2023.

(51) Int. Cl. A61B 5/00; A61B 5/145; A61B 5/1486; A61B 5/1495

(52) U.S. Cl. CPC A61B 5/4266; A61B 5/14532; A61B 5/1486; A61B 5/1495; A61B 5/7455; A61B 2560/0238; A61B 2562/0247

(57) ABSTRACT

Provided herein are methods, devices, and systems that pertain to solid-state sensor and sensor-reading meter for touch-based rapid physiological and chemical sensing.

### CROSS-REFERENCE

This application is a continuation of International Application No. PCT/US2024/036816, filed Jul. 3, 2024, which claims the benefit of U.S. Provisional Application No. 63/512,248, filed Jul. 6, 2023, each of which are incorporated herein by reference in their entirety.

### BACKGROUND

Sweat contains a number of small-molecule biomarkers and may be a prospective alternative biofluid to blood and interstitial fluid for non-invasive sensing. However, sweat-based sensing may be limited in accuracy and practicality, because these methods often require an additional step of sweat generation and collection process, has long delay compared to blood-based sensing, and may be influenced by individual differences and sweat rates. As such, there is a need for accurate, convenient, rapid, and non-invasive biomarker sensing while taking advantage of sweat as an easily accessible sample to measure various biomarkers.

### SUMMARY

In an aspect, the present disclosure provides a system, comprising: a test strip comprising a solid-state electrochemical sensor layer and a conductive layer; and a test strip reading device comprising a slot, configured to receive a first portion of the test strip and to keep a surface of a second portion of the test strip uncovered, wherein the uncovered surface is accessible to contact a portion of skin of a user.

In some embodiments, the system further comprises a computer processor operably coupled to the test strip reading device configured to use the test strip reading device to determine a property of the skin. In some embodiments, the test strip reading device comprises a physical data port. In some embodiments, physical data port is a universal serial bus (USB) port. In some embodiments, the USB port is configured to permit communication between the test strip reading device and a computing system. In some embodiments, the USB port is configured to receive power from a computing system when connected to the computing system. In some embodiments, the test strip reading device comprises a wireless communication module. In some embodiments, the wireless communication module comprises a Bluetooth® communication module, a Wi-Fi® communication module, a near field communication (NFC) module, a cellular communication module, a radio communication module, an ultra wideband communication module, or any combination thereof. In some embodiments, the test strip reading device is reusable. In some embodiments, the test strip is reusable. In some embodiments, the test strip is reusable for at least about 3 days. In some embodiments, the test strip is reusable for at least about 7 days. In some embodiments, the test strip is reusable for at least about 15 uses. In some embodiments, the test strip is not reusable. In some embodiments, the test strip reading device is not reusable. In some embodiments, said test strip reading device is configured to, after testing said skin, permit removal of said test strip. In some embodiments, after said removal of said test strip, said test strip reading device is configured to receive an additional test strip. In some embodiments, the test strip reading device is configured to be affixed to a computing device. In some embodiments, the computing device is a mobile phone, a watch, a wearable device, or a laptop computer. In some embodiments, the test strip reading device comprises a microprocessor configured to interface with the test strip to take a measurement from the test strip. In some embodiments, the system further comprises a lid positioned to protect the uncovered surface of the test strip. In some embodiments, the lid is slidable with respect to the test strip reading device. In some embodiments, the lid is attached to the test strip reading device using a hinge. In some embodiments, the lid comprises one or more sensors configured to determine an open or closed state of said lid. In some embodiments, the test strip reading device has a volume of at most about 4 cubic centimeters (cm³). In some embodiments, the test strip reading device is configured to guide a finger of said user to said test strip. In some embodiments, the system further comprises a display in the test strip reading device. In some embodiments, the system further comprises one or more physical buttons. In some embodiments, the test strip reading device comprises one or more sensors. In some embodiments, said one or more sensors are configured to acquire one or more signals comprising a temperature of the user, a temperature of the test strip reading device, a moisture level of the user, a humidity, a sweat gland density of the user, a sweat rate of the user, a transdermal water loss rate of the user, pressure, a skin hydration level of the user, a heart rate of the user, a blood oxygen level of the user, a blood pressure of the user, a skin conductance of the user, a skin capacitance of the user, a skin resistance of the user, a skin impedance of the user, or any combination thereof. In some embodiments, said test strip reading device has a credit card form factor, a keychain form factor, or a ring form factor. In some embodiments, said test strip reading device is integrated in a laptop. In some embodiments, said test strip reading device is configured to be integrated into or associated with a mobile telephone case. In some embodiments, the test strip reading device is configured to be covered by a portion of the mobile telephone case.

In another aspect, the present disclosure provides a method, comprising: (a) providing a test strip reading device holding a test strip comprising a solid-state electrochemical sensor layer and a conductive layer; (b) contacting the test strip with a portion of a subject's skin; (c) using the solid state electrochemical sensor layer and the conductive layer to generate an electrical measurement based on a biomarker from the portion of the subject's skin or sweat perspired thereon; and (d) using the electrical measurement to determine a property of the biomarker of the subject.

In some embodiments, the property of the biomarker is a blood glucose level of the subject. In some embodiments, the method further comprises using a calibration measurement in the determining of the property of the biomarker. In some embodiments, the determining of the property of the biomarker is performed without a pre-calibration. In some embodiments, the method further comprises, prior to (b), opening a lid affixed to the test strip reading device to expose the test strip. In some embodiments, the test strip is reusable. In some embodiments, the test strip is reusable at least about 15 times.

Provided herein are methods, devices, and systems designed for touch-based rapid physiological and chemical sensing.

In some embodiments, the system includes a portable, sweat-based analyte monitoring system. In some embodiments, the sensor device includes a test strip comprising a solid-state electrochemical sensing layer and a conductive layer; and a test strip reading device, wherein the test strip is configured to contact a portion of skin of a user to receive a sample from the skin, and wherein the test strip reading device is configured to receive the test strip. In some embodiments, the sample comprises sweat.

In some embodiments, the conductive layer comprises a current conducting material. In some embodiments, the current conducting material comprises one or more of a metal, a doped ceramic, a carbonaceous material, or a conductive polymer. In some embodiments, the metal comprises one or more of gold, silver, copper, ruthenium, rhodium, platinum, bismuth, tungsten, iron, titanium, rhenium, osmium, or iridium. In some embodiments, the doped ceramic comprises one or more of indium tin oxide, aluminum-doped zinc oxide, or fluorine-doped tin oxide. In some embodiments, the carbonaceous material comprises one or more of graphite, carbon black, carbon nanotubes, graphene, or reduced graphene oxide. In some embodiments, the conductive polymer comprises one or more of poly(3,4-ethylenedioxythiophene) polystyrene sulfonate, polypyrrole, polyaniline, polythiophene, or poly(p-phenylene), or poly phenylenediamine.

In some embodiments, the conductive layer is configured to conduct an electrochemical signal and establish an electrical connection to the test strip reading device when the test strip is inserted into the test strip reading device. In some embodiments, the sensor comprises an electrochemical transducer comprising a biorecognition functionality. In some embodiments, the sensor comprises one or more of an enzyme, an ionophore, a binding polypeptide, a polynucleotide, an aptamer, a molecularly imprinted polymer, or a microbe. In some embodiments, the enzyme comprises one or more of glucose oxidase, glucose dehydrogenase, alcohol oxidase, alcohol dehydrogenase, lactate oxidase, lactate dehydrogenase, cholesterol oxidase uricase, urease, ascorbate oxidase, horseradish peroxidase, catalase, tyrosinase, creatinine deiminase, amylase, glutamate oxidase, xanthine oxidase, bilirubin oxidase, hydroxybutyrate dehydrogenase, hydroxybutyrate oxidase, D-amino acid oxidase, L-amino acid oxidase, pyruvate oxidase, or acetoacetate dehydrogenase. In some embodiments, the binding polypeptide comprises an antibody or an antigen binding fragment.

In some embodiments, the sensor further comprises one or more of a cofactor, a mediator, a stabilizer, a surfactant, a polymer, a plasticizer, a cross-linker, a cross-linking terminator, or a cross-linking initiator. In some embodiments, the cofactor comprises one or more of nicotinamide adenine dinucleotide, nicotinamide adenine dinucleotide phosphate, flavin adenine dinucleotide, flavin mononucleotide, thiamine pyrophosphate, biotin, heme, coenzyme A, coenzyme Q, cobalamin, pyridoxal phosphate, tetrahydrofolic acid, or S-adenosyl methionine. In some embodiments, the mediator comprises one or more of ferrocene and ferrocene derivatives, ferricyanide, ferrocyanide, Prussian blue, quinones, tetrathiafulvalene, organic dyes (methylene blue, Meldola blue), osmium complex, or ruthenium complex. In some embodiments, the stabilizer comprises one or more of glycerol, trehalose, chitosan, albumin, polyethylene glycol, calcium chloride, silica, polyols, diethyldithiocarbamate, or mercaptoundecanol. In some embodiments, the surfactant comprises a Pluronic triblock copolymer. In some embodiments, the polymer comprises one or more of alginate, chitosan, acrylate, methacrylate, or saccharides. In some embodiments, the polymer comprises a monomer of the polymers. In some embodiments, the plasticizer comprises one or more of di-2-ethylhexyl phthalate, diisononyl phthalate, dioctyl adipate, diisononyl adipate, triphenyl phosphate, polyethylene glycol, dibutyl sebacate, dioctyl sebacate, acetyl tributyl citrate, or epoxidized soybean oil. In some embodiments, the cross-linker comprises one or more of glutaraldehyde, 1-ethyl-3-(3-dimethylaminopropyl)carbodiimide, N-hydroxysuccinimide, polyethylene glycol diglycidyl ether, glyoxal, benzophenone, disuccinimidyl suberate, bis(sulfosuccinimidyl) suberate, or dithiobis(succinimidyl propionate). In some embodiments, the cross-linking initiator comprises one or more of azobisisobutyronitrile, potassium persulfate, ammonium persulfate, tetramethylethylenediamine, benzoyl peroxide, or Irgacure 2959.

In some embodiments, the test strip further comprises a substrate. In some embodiments, the substrate comprises one or more of plastics, ceramics, or natural materials. In some embodiments, the substrate comprises one or more of polyethylene, polypropylene, polyethylene terephthalate, polyester, polyimide, polydimethylsiloxane, alumina, silicon, or paper. In some embodiments, the substrate is treated mechanically, chemically, and/or physically to change a characteristic. In some embodiments, the characteristic comprises one or more of surface roughness, hydrophobicity, dielectric constant, or electrostatic charge. In some embodiments, the substrate treatment comprises one or more of base chemical etching, plasma etching, electroplating, abrasion, laser ablation, sputtering, heating, and physical or chemical vapor deposition. In some embodiments, substrate treatment improves adhesion of the sample and/or compatibility of the sample.

In some embodiments, the test strip further comprises an insulation layer. In some embodiments, the insulation layer comprises a non-conductive material. In some embodiments, the insulation layer is waterproof In some embodiments, the insulation layer is rigid. In some embodiments, the insulation layer is transparent. In some embodiments, the insulation layer has an antibacterial property. In some embodiments, the insulation layer covers a portion of the test strip to define an exposed area for contacting the skin. In some embodiments, the insulation layer is deposited or attached to another layer of the test strip.

In some embodiments, the test strip further comprises a counter-interference layer. In some embodiments, the counter-interference layer comprises one or more of a metal, a mediator, a negatively or positively charged molecule. In some embodiments, the mediator comprises one or more of ferrocene and ferrocene derivatives, ferricyanide, ferrocyanide, Prussian blue, quinones, tetrathiafulvalene, an organic dye, an osmium complex, or a ruthenium complex. In some embodiments, the organic dye comprises one or more of methylene blue or Meldola blue. In some embodiments, the negatively or positively charged molecule comprises one or more of Nafion, polychlorotrifluoroethylene, polyfluoroalkanes, fluorine rubber, chitosan, polyethyleneimine, polyurethane, poly(p-phenylenediamine), polystyrene sulfonate, polyvinyl sulfate, polyvinyl alcohol, or polyvinyl chloride. In some embodiments, the fluorine rubber comprises one of more of copolymer of vinylidene fluoride, tetrafluoroethylene, hexafluoropropylene, and perfluoromethylvinylether. In some embodiments, the fluorine rubber comprises the addition of ethylene and propylene in the copolymer.

In some embodiments, the test strip further comprises a protective layer. In some embodiments, the protective layer comprises a polymer. In some embodiments, the polymer comprises one or more of Nafion, polychlorotrifluoroethylene, chitosan, methyl- or ethyl-cellulose, polyvinyl chloride, polyurethane, poly(p-phenylenediamine), silicone, polytetrafluoroethylene, polyolefin, polyester, polycarbonate, copolymers, polyvinylidene fluoride, polymethyl methacrylate, polyvinyl alcohol, polyethylene glycol, poly acrylamide, polyacetate, polyvinylpyrrolidone, polyethylene oxide, or polysulfides. In some embodiments, the protective layer is permeable. In some embodiments, the protective layer is configured to protect the layers beneath from mechanical and/or chemical damage.

In some embodiments, the test strip further comprises a conditioning layer. In some embodiments, the conditioning layer comprises a polymer and a surfactant. In some embodiments, the conditioning layer is configured to adjust hydrophilicity and electrostatic charge of the electrode. In some embodiments, the test strip is single-use. In some embodiments, the test strip is reusable for at least 10 uses.

In some embodiments, a layer is patterned for improved sweat contact and signal transduction. In some embodiments, the pattern comprises one or more of an interdigitated pattern, a concentric pattern, a radially interdigitated pattern. each segment of a line or an arc of the pattern is about 1 mm away from each other. In some embodiments, the analyte comprises one or more of sodium, potassium, calcium, magnesium, chlorides, fluorides, glucose, lactates, alcohols (ethanol), ketones (β-hydroxybutyrate, acetoacetate), cortisol, uric acid, urea, ascorbates, creatinine, creatine, amino acids (e.g., glycineleucine, proline, lysine, alanine, glutamine, tyrosine, tryptophan, cysteine, etc.), levodopa, caffeine, cannabinoids, cocaine, opioids, explosives, or nerve agents.

In some embodiments, the device comprises a housing and a circuit board. In some embodiments, the circuit board comprises a microcontroller, a signal generation and processing chip, a data storage chip, and a connector configured to receive the test strip. In some embodiments, the circuit board further comprises one or more of a wireless transmission functionality, an antenna, a display panel, an indicator light, a user input button, or a power source. In some embodiments, the user input button comprises an electrical button or a mechanical button or a combination thereof. In some embodiments, the circuit board is configured to generate an electrochemical measurement when the test strip is inserted into the device and the sample is contacted onto the test strip. In some embodiments, the electrochemical measurement comprises one or more of open circuit voltage measurement, chronopotentiometry, chronoamperometry, chronocoulometry, cyclic voltammetry, differential pulse voltammetry, square wave voltammetry, linear sweep voltammetry, alternating current impedance spectrometry, or direct current internal resistance measurement. In some embodiments, the circuit board is configured to generate an electrical signal from an electrochemical measurement from the sample and process the electrical signal to determine a concentration of the analyte. In some embodiments, the circuit board processes the electrical signal into the concentration of the analyte using a pre-programmed algorithm and a user-input calibration data. In some embodiments, the pre-programmed algorithm comprises a two-point or single-point linear regression using the user-input calibration data. In some embodiments, the user-input calibration data is obtained from another analyte monitoring device. In some embodiments, the another analyte monitoring device comprises a blood glucose monitor or a continuous glucose monitor. In some embodiments, the pre-programmed algorithm is personalized to the user. In some embodiments, the pre-programmed algorithm is individualized for different fingers of the user. In some embodiments, the data storage chip is configured to store one or more of a number of uses of the test strip, a raw signal, processed data, time of measurement, or the personalized and individualized calibration data. In embodiments, the analyte is referred herein as a small molecule biomarker.

In some embodiments, the circuit board is configured to deliver the stored information to a secondary device. In some embodiments, the secondary device comprises one or more of a computer, a mobile device, or a cloud-based data center. In some embodiments, the secondary device performs data analysis and data visualization on the stored information. In some embodiments, the device further comprises one or more of an optical sensor or an electrical sensor. In some embodiments, the optical sensor or the electrical sensor is configured to measure one or more physiological and physical signals from the skin. In some embodiments, the other one or more physiological and physical signals comprises one or more of temperature, moisture level, sweat gland density, sweat rate, transdermal water loss rate, pressure, hydration level, heart rate, skin conductance, skin capacitance, skin resistance, skin impedence, or blood oxygen level. In some embodiments, the pre-programmed algorithm uses the other one or more physiological and physical signals to make a correction in the calculation of the analyte concentration from the electrical signal. In some embodiments, the device further comprises an optical, a capacitive, or an ultrasonic fingerprint scanner to identify the user and the finger contacting the test strip.

In some embodiments, the housing is configured to protect the circuit board and the test strip when inserted into the device. In some embodiments, the housing comprises a moving cover to cover a portion of the test strip inserted into the device when the moving cover is closed and to uncover the portion of the test strip when the moving cover is opened. In some embodiments, the cover comprises a replaceable wipe or a squeegee to remove the sample residue after contact with the skin. In some embodiments, the housing comprises a replaceable wiper for wiping the skin prior to contacting the test strip. In some embodiments, the housing comprises a mechanical guide for contact by the skin.

Provided herein are methods of non-invasively detecting an analyte from a fingertip, the method comprising: inserting the test strip of any one of preceding claims into the device of any one of preceding claims; placing a portion of skin of a user in contact with the test strip; and obtaining a reading of the analyte from the test strip reading device. In other embodiments, provided herein are methods of non-invasively monitoring an analyte from a fingertip, the method comprising: receiving a sample from a skin of a user on the test strip of any one of preceding claims inserted into the device of any one of preceding claims by contact of the skin to the test strip; generating an electrical signal from an electrochemical measurement of the analyte using the solid-state electrochemical sensor; measuring a concentration of the analyte in the sample from the electrical signal; calculating, by the device, a systemic concentration of the analyte in the user from the concentration of the analyte in the sample; and providing the systemic concentration of the analyte on a display of the device.

Provided herein are methods of repairing a test strip for a sweat-based analyte monitoring system, the method comprising: deposition of one or more layers on top of a substrate; and attaching the deposited layer to the substrate or the previous layer, wherein the test strip comprises the substrate, a conductive layer, a solid-state electrochemical sensor, and an insulation layer. In some embodiments, the deposition comprises one or more of electrodeposition, sputtering, physical and chemical vapor deposition, screen printing, transfer printing, dip coating, spray coating, spin coating, doctor blading, inkjet printing, drop casting, or microdot patterning. In some embodiments, the method of attaching the layer comprises one or more of cold pressing, hot pressing, adhesion, crimping, or embedding. In some embodiments, a layer is patterned to improve sweat contact and signal transduction. patterning comprises one or more of direct deposition or printing, masked deposition or printing, masked etching, laser ablation, laser cutting, mechanical abrasion, or mechanical cutting.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an exploded perspective view of one exemplary embodiment of electrochemical solid-state sensor.

FIG. 2A shows chemical or physical treatment process of substrate along with certain amount of temperature. FIG. 2B shows laser ablation of metal sputtered substrate.

FIGS. 3A(i)-3B(ii) show signal comparison of with and without counter-interference layer to common interference species (acetaminophen (AP), uric acid (UA), and ascorbic acid (AA)).

FIGS. 4A-4C show sensitivity comparison towards glucose of with and without substrate treatment along with stability test for multiple repeated experiments.

FIG. 5 shows a schematic illustration of a sensor with application of finger to the sensor for collection of sweat and biomarkers therein.

FIG. 6 shows a schematic of the system combining with biosensor and housing device.

FIG. 7 shows a schematic of the test strip for insertion into the device,

FIG. 8 shows a schematic of the system combining with the electrochemical sensing test strip, device and batteries.

FIG. 9 shows a schematic of the cover for opening/closing the notch holder for the test strip.

FIGS. 10A-10B show examples of a credit card form factor test strip reading device.

FIGS. 11A-11C show examples of a key chain form factor test strip reading device.

FIGS. 12A-12B show examples of a laptop form factor test strip reading device.

FIGS. 13A-13C show examples of a mobile phone case form factor test strip reading device.

FIGS. 14A-14C show examples of a ring form factor test strip reading device.

FIGS. 15A-15C show examples of a universal serial bus plug in form factor test strip reading device.

FIGS. 16A-16C show examples of a smart watch band form factor test strip reading device.

FIGS. 17A-17B show examples of a handheld form factor test strip reading device.

FIGS. 18A-18B show examples of a pressure switch and an integration into a test strip reading device.

FIGS. 19A-19C show examples of a pressure switch in activation and in a test strip reading device.

FIG. 20 shows an example of an exploded view of a test strip reading device.

### DETAILED DESCRIPTION

Sweat contains various small-molecule biomarkers and may be a prospective alternative biofluid to blood and interstitial fluid for non-invasive sensing. Sweat biomarker sensing may use a combination of a biosensor with high specificity to the target analytes, a sweat generation and collection mechanism, and an electronic device for signal generation, data processing, and human interfacing.

Previous sweat biomarker monitoring systems may have relied on exercise-based sweat, transdermal drug-delivery-induced sweat, or heat-induced sweat that is collected using microfluidic mechanisms or hydrogels for subsequent reaction with the signal transducing sensor electrodes. Such methods may be limited in accuracy and practicality, as they often require extended and arduous sweat generation and collection process, has long delay compared to blood-based biomarker monitoring, and are heavily influenced by individual differences and sweat rates.

Recent fingertip touch-based sensors may allow for sweat collection from the fingertip without the need for exercise or active extraction using hygroscopic porous hydrogel for analyte collection and the electrolyte that covers the electrode surfaces to enable rapid non-invasive sensing. However, such sensors can still require the use of hydrogel that is prone to drying, and contain additional problems on the sensing process due to analyte dilution and accumulation. The use of hydrogel is difficult in its execution and storage, making the device and operation less practical and accessible for users.

In an aspect, the present disclosure provides a novel portable, integrated sweat monitoring system comprising two main components: a solid-state electrochemical test strip and an electronic device into which the test strip can insert. The system can be used for, for example, non-invasive sweat sensing, where the user is to insert the test strip into the electronic device, and place their finger onto the test strips for a given amount of time, during which electrochemical measurement will be performed by the electronic device to the test strip to generate raw electrical signals; the electrical signals will be thereafter processed by the electronic device and generate the desired results, such as the concentration of the target analyte.

Disclosed herein are methods, devices, and systems that pertain to solid-state gel-free sensor for touch-based rapid physiological and chemical sensing. The disclosed technology can be implemented in some embodiments to provide a new class of non-invasive, pain-free sensors for the direct sampling and frequent measurement of the biomarker in sweat and the related physiological states, such as sweat rates.

The novel portable sweat monitoring system provided herein may provide various advantages, including but not limited to a more accurate, convenient, rapid, and non-invasive biomarker sensing. The system obviates the need for sweat stimulation and instead uses natural fingertip perspiration as the biofluid for sensing, which can be highly advantageous. It can comprise two main components: a disposable electrochemical sensing test strip and a reusable electronic meter. The unique design and composition of the test strips allow the detection and quantification of biomarkers in a very low amount of natural fingertip perspiration (<100 nL), unaffected by sweat rates, chemical and physical interferences, and environmental factors. The electronic meter may have a specialized signal generation and conversion algorithms, along with unique mechanical design considerations to increase user friendliness and limit human errors during its usage.

### Electrochemical Sensing of Biomarkers

### A. Sweat Based Monitoring System

In some embodiments, the system can include a portable, sweat-based analyte monitoring system. In some embodiments, the sensor device includes a test strip comprising a solid-state electrochemical sensor layer and a conductive layer; and a test strip reading device, wherein the test strip is configured to contact a portion of skin of a user to receive a sample from the skin, and wherein the test strip reading device is configured to receive the test strip. the sample comprises sweat.

In some embodiments, the sensor can operate with the user's direct contact using a fingertip or any other skin surfaces that emit passive, natural, thermoregulatory eccrine sweat, and measure the concentration of various ions and biomolecules in the sweat (e.g., sodium, potassium, chloride, glucose, lactate, urea, uric acid, bilirubin, hydroxybutyrate, vitamins, amino acids, alcohol, levodopa, caffeine, cortisol, insulin, explosives, narcotics, nerve agents, fluoride, calcium, zinc, lead, cadmium, mercury) via electrical (e.g., conductivity, piezoresistive, thermoresistive, piezocapacitive, thermoelectric, piezoelectric), chemical (e.g., non-specific adsorption, specific binding, intercalation, insertion), or electrochemical (e.g., catalysis, redox reaction) transduction methods.

Diabetes patients that need constant glucose monitoring can use finger-pricking blood glucose meters which may be highly invasive and painful. Alternative continuous glucose monitoring systems can still requires the insertion of needles into the body for continuous sensing, which is invasive and requires maintenance. For the sensing of glucose and many other biomarkers important to human health, new wearable sensors can be used, featuring non-invasive sensing of more accessible biofluids such as sweat or interstitial fluids. The use of such epidermal sensors may require arduous analyte extraction processes such as exercises, heat, or iontophoresis, and rely on complicated device structure, such as microfluidic devices, microneedles, iontophoretic patch etc. Such processes can be intrusive, complex, and high maintenance for self-monitoring. Moreover, these devices may require either a large quantity of analyte, or require special analyte uptake mechanism, such as microfluidic channels, or hydrogels, to ensure coverage of the electrode surfaces.

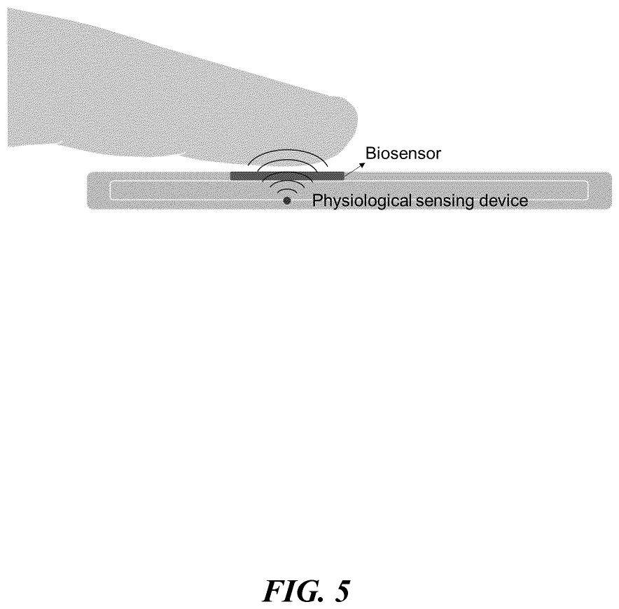

Fingertip touch-based can enable sweat collection from the fingertip without the need for exercise nor active extraction using hygroscopic porous hydrogel for analyte collection and the electrolyte that covers the electrode surfaces to enable rapid non-invasive sensing and can be used for the sensing of various chemicals. However, this process may still requires the use of a hydrogel that is prone to drying, and can contain additional problems on the sensing process due to analyte dilution and accumulation. The use of a hydrogel may be difficult in its execution and storage, making the device and operation less practical and accessible for users. FIG. 5 show examples of a test strip with a sensor implemented based on some embodiments of the disclosed technology. FIG. 5 shows a fingertip in contact with a sensor on a test strip as implemented based on some embodiments of the disclosed technology and is an example of gel-free sensor for touch-based rapid physiological and chemical sensing implemented based on some embodiments of the disclosed technology. Embodiments of the disclosed technology can obviate the need for external analyte collection mechanisms, such as the use of hydrogels, hydrocolloids, porous materials, microfluidic channels, microneedles, iontophoresis, reverse iontophoresis, transdermal cholinergic agent delivery etc., and allows rapid, maintenance-free user-friendly near real-time biochemical and physiological sensing.

In embodiments, the technology here can be implemented using closely spaced electrodes functionalized with transducers for biomarkers, wherein the user can perform sensing non-invasively, painless, and maintenance-free in a rapid fashion. The closely spaced or interdigitated electrode design can obviate the use of hydrogel, which make the device more accessible, simple, stable, and for frequent repetitive measurements. The disclosed technology can be implemented in some embodiments to provide an approach for epidermal sweat sensing that obviates the need for any arduous sweat collection process such as exercises, heat, chemical stimulation or iontophoretic extraction.

A single sensor strip can be used reliably for repeated multiple glucose testing to allow the users to track their glucose level and detect potential glycemic abnormalities conveniently and closely. An initial first-day, simple 2-data-point personalized calibration (at different blood glucose levels) can be used to fully address inter-person variations (e.g., sweat rate). The sensor can deliver highly accurate blood glucose concentration data, closely matching the capillary blood glucose (CBG) level of commercial fingerstick.

The design thus can enable the solid-state contact-based fingertip sensing, offering significant advantages over hydrogel-based sweat collection mechanisms in terms of simplicity, reusability, testing frequency, and data reproducibility. As a comparison, the same finger can be tested repeatedly on the same sensor with and without the hydrogel with steady and falling glucose levels (before and 30 min after a meal, respectively). In the case of constant glucose level, the hydrogel-covered sensor can display an increasing current signal due to the carry-over and build-up of glucose from repeated touches, while the solid-state sensor shows good stability, with negligible difference through these repeated touches. In contrast, in the case of a falling glucose level, the solid-state sensor can accurately capture the dynamically decreasing glucose concentrations, whereas the hydrogel-covered sensor shows a slowly increasing response due to the combination of competing effects of the decreasing glucose concentration and its buildup in the gel, leading to unrealistically increasing response associated with such carry-over effects. The reproducibility of the sensor at different glucose concentrations can be investigated from five repeated tests at the fasting state.

The disclosed technology can be implemented in some embodiments to provide a highly accurate, simple, and rapid glucose-sensing protocol using an interdigitated, solid-state PEDOT:PSS-based electrode, which leverages the passive perspiration of the fingertips to enable reliable near real-time non-invasive monitoring of glucose levels. Eliminating the need for the sweat collecting hydrogel interface can greatly simplify the operation to allow frequent repetitive measurements over the entire day while offering superior analytical performance compared to traditional hydrogel-based SPE sensors. Compatible with diverse subjects, the sensor can rapidly establish personalized calibration (from merely initial 2 fingerstick measurements), showing considerable promise towards substituting painful, frequent finger pricking SMBG and invasive CGM technologies for extended day-long continuous glucose monitoring. The new protocol may offer high accuracy with low MARD and good CEGA (Clarke Error Grid Analysis) metrics, which are comparable to those of commercial glucose sensing technologies, along with painless (blood- and needle-free), rapid operation.

The same low-cost sensor can be used without any re-stabilization. Such convenient touch-based sensing can greatly increase the frequency of self-testing compared to traditional SMBG, to offer enhanced diabetes control. The disclosed technology may also be implemented in some embodiments to enable large-scale validation using diverse subjects, and to increase further the speed and simplicity develop an advanced blood-free calibration process and prediction algorithm, along with improved understanding of the fingertip passive perspiration phenomenon and of the role of the electrode geometry at the hydrogel-free (Interdigitated Electrode) setup. Translating such contact-based sensing towards passive, repeated monitoring of glucose through the day can further promote its practical usage as a true CGM alternative.

Combined with engineering efforts for creating a user-friendly sensor prototype, these developments may lead to highly reliable pain-free rapid, and frequent self-testing of glucose for home and other decentralized settings towards improved management of diabetes, as well as for simplified and accurate non-invasive monitoring of other key sweat biomarkers, for example, ketones, cortisol, drug-like compounds etc.

### Test Strip Assembly

The test strip can comprise at least one substrate. In some cases, the substrate comprises one or several types of plastics, ceramics, or natural materials, such as polyethylene, polypropylene, polyethylene terephthalate, polyester, polyimide, polydimethylsiloxane, alumina, silicon, and paper. In some cases, the sensor includes a substrate made of glasses, silicon, paper, textile, or polymeric plastics or elastomers. Also, such test strips can be fabricated or integrated on other platforms, for example, the substrate can include silicone or polyurethane membranes, e.g., such as a polyurethane-based elastomer.

In some cases, the surface of the substrate can be mechanically, chemically, or physically treated to change its surface roughness, cleanness, hydrophobicity, dielectric constant, and electrostatic charge for improved adhesion and compatibility with sweat sensing. In some cases, the treatment process includes but is not limited to: acid or base chemical etching, plasma etching, electroplating, abrasion, laser ablation, sputtering, heating, and physical or chemical vapor deposition.

In some embodiments, the test strip comprises an electrochemical sensor layer. The electrochemical sensing layer can include at least an electrochemical transducer with biorecognition functionalities, such as enzymes (glucose oxidase, glucose dehydrogenase, alcohol oxidase, alcohol dehydrogenase, lactate oxidase, lactate dehydrogenase, cholesterol oxidase uricase, urease, ascorbate oxidase, horseradish peroxidase, catalase, tyrosinase, creatinine deiminase, amylase, glutamate oxidase, xanthine oxidase, bilirubin oxidase, hydroxybutyrate dehydrogenase, hydroxybutyrate oxidase, acetoacetate dehydrogenase), ionophores, antibodies, nucleic acids, aptamers, molecularly imprinted polymers, or microbes.

The electrochemical sensing layer can include one or multiple cofactors such as nicotinamide adenine dinucleotide, nicotinamide adenine dinucleotide phosphate, flavin adenine dinucleotide, flavin mononucleotide, thiamine pyrophosphate, biotin, heme, coenzyme A, coenzyme Q, cobalamin, pyridoxal phosphate, tetrahydrofolic acid, S-adenosyl methionine.

The electrochemical sensing layer can include one or multiple mediators such as ferrocene and ferrocene derivatives, ferricyanide, ferrocyanide, Prussian blue, quinones, tetrathiafulvalene, organic dyes (methylene blue, Meldola blue), osmium or ruthenium complexes.

The electrochemical sensing layer can include one or multiple stabilizers such as glycerol, trehalose, chitosan, albumin, polyethylene glycol, calcium chloride, silica, polyols, diethyldithiocarbamate, mercaptoundecanol), or surfactants such as Pluronic triblock copolymers. Examples of surfactants include, but are not limited to, fluorosurfactants (e.g., surfactants comprising fluorine moieties), surfynol (e.g., tetramethyldecynediol), triton-X 100, triton-X 114, or the like.

The electrochemical sensing layer can include one or multiple polymers or monomers of polymers (alginate, chitosan, acrylate, methacrylate, saccharides), plasticizers (di-2-ethylhexyl phthalate, diisononyl phthalate, dioctyl adipate, diisononyl adipate, triphenyl phosphate, polyethylene glycol, dibutyl sebacate, dioctyl sebacate, acetyl tributyl citrate, epoxidized soybean oil).

The electrochemical sensing layer can include one or multiple cross-linkers such as glutaraldehyde, 1-ethyl-3-(3-dimethylaminopropyl)carbodiimide, N-hydroxysuccinimide, polyethylene glycol diglycidyl ether, glyoxal, benzophenone, disuccinimidyl suberate, bis(sulfosuccinimidyl) suberate, dithiobis(succinimidyl propionate).

The electrochemical sensing layer can include one or multiple and cross-linking initiators such as azobisisobutyronitrile, potassium persulfate, ammonium persulfate, tetramethylethylenediamine, benzoyl peroxide, Irgacure 2959. The electrochemical sensing layer may comprise one or more cross-linking terminators (e.g., lysine, glycine, poly-lysine, etc.).

In some embodiments, the test strip comprises at least a conductive layer. In some cases, the conductive layer serves the purpose of conducting electrochemical signals and establishing electrical connections to the electronic device upon inserting. In some cases, it is composed of current conducting materials, such as one or multiple types of metal (gold, silver, copper, ruthenium, rhodium, platinum, bismuth, tungsten, iron, titanium, rhenium, osmium, iridium), doped ceramics (indium tin oxide, aluminum-doped zinc oxide, fluorine-doped tin oxide), carbonaceous materials (graphite, carbon black, carbon nanotubes, graphene, reduced graphene oxide), and conductive polymers (poly(3,4-ethylenedioxythiophene) polystyrene sulfonate, polypyrrole, polyaniline, polythiophene, poly(p-phenylene), poly phenylenediamine, or 2D materials (e.g., MoS2, WSe2, VO2).

In some cases, the layer can comprise electrically semi-conductive material with added impurities to change its resistive properties. For example, the electrically semi-conductive material can include a semi-conductive ink, e.g., including, but not limited to, amorphous carbon, carbon black, graphite, carbon nanotubes, and/or graphene. In some embodiments of the method, for example, the electrode pattern can include carbon fiber segments dispersed within the electrically conductive or electrically semi-conductive material.

The test strip may comprise a permeable protective layer to protect the layers beneath from mechanical or chemical damage. The protective layer can comprise one or a combination of polymers (Nafion, chitosan, methyl- or ethyl-cellulose, polyvinyl chloride, polyurethane, silicone, polytetrafluoroethylene, polyolefin, polyester, polycarbonate, copolymers, polyvinylidene fluoride, polymethyl methacrylate, and polysulfides). The sensor may also include a protection layer composed of polymeric materials such as Nafion, chitosan, ethylcellulose, polyvinyl chloride, polyvinyl alcohol, polyethylene glycol, poly acrylamide, polyacetate, polyvinylpyrrolidone, or polyethylene oxide.

The test strip may comprise a counter-interference layer, which comprises one or a combination of metals, mediators (ferrocene and ferrocene derivatives, ferricyanide, ferrocyanide, Prussian blue, quinones, tetrathiafulvalene, organic dyes (methylene blue, Meldola blue), osmium or ruthenium complexes), negative/positive charged molecules or polymers (Nafion, polyfluoroalkanes, fluorine rubber, chitosan, polyethyleneimine, polyurethane, polystyrene sulfonate, polyvinyl sulfate, polyvinyl alcohol, polyvinyl chloride). In some embodiments, the fluorine rubber comprises one of more of copolymer of vinylidene fluoride, tetrafluoroethylene, hexafluoropropylene, and perfluoromethylvinylether. In some embodiments, the fluorine rubber comprises the addition of ethylene and propylene in the copolymer. In some embodiments, the fluorine rubber is a elastomer.

The sensor may include an insulation layer composed of dielectric materials. The insulation layer covers the test strips partially to define and limit the exposed area and can be deposited or attached to the previous layers. The insulation layer can be made with a non-conductive material and may possess specific desired properties, such as waterproof, rigid, transparent, or antibacterial.

The test strip may comprise a conditioning layer to adjust the hydrophilicity and electrostatic charge of the electrode. The conditioning layer can comprise one of a combination of polymers and surfactants.

In some cases, the test strip can be reusable (e.g., capable of being used for multiple readings). The test strip may be reusable at least about 2, 3, 4, 5, 6, 7, 8, 9, 10, 11, 12, 13, 14, 15, 16, 17, 18, 19, 20, 21, or more times. The test strip may be reusable for at least about 1, 2, 3, 4, 5, 6, 7, 8, 9, 10, 11, 12, 13, 14, or more days. The test strip may be disposable (e.g., not reusable). For example, the test strip may be discarded after each use. The test strip reading device may be reusable. For example, the test strip reading device may be configured to permit removal of a used test strip and have a new test strip replaced into the test strip reading device. In this way, the test strip reading device can be reused as a housing and interface for a plurality of different test strips. The test strip reading device may be reused for at least about 2, 5, 10, 20, 30, 40, 50, 60, 70, 80, 90, 100, or more test strips. A reusable test strip reading device may provide advantages such as lower long-term costs, increased sophistication and capability of onboard sensors and computational power, convenience for a user, or the like. In some cases, the test strip reading device may not be reusable. For example, the test strip reading device may be configured to only accept a single test strip and, when that test strip is spent, be discarded along with the test strip. A disposable test strip reading device may provide lower upfront costs, smaller form factors, or the like.

In some cases, the test strip reading device can be configured to, after testing a sample, permit removal of a test strip. For example, the test strip may not be permanently affixed to the test strip reading device. The test strip may interface with the test strip reading device by, for example, electronic leads (e.g., pogo pins, conductive clamping leads, etc.). The test strip reading device may comprise a portion configured to hold the test strip in place as well as facilitate the connection of the test strip to the electronics of the test strip reading device. In some cases, the test strip reading device can be configured to accept a test strip (e.g., after removal of an expired test strip). In this way, the test strip reading device can be reusable for a plurality of test strips as described elsewhere herein.

The test strip reading device may be configured to read information from a test strip. The information may be read through a physical connection (e.g., via contacts). The information may be read wirelessly (e.g., through a near field communication module). The information may comprise, for example, a type of test strip (e.g., identify the biomarker the test strip tests for), a batch number or lot number of the test strip, a remaining number of measurements the test strip can be used for, a health of the test strip (e.g., a presence or absence of defects in the test strip), a cleanliness of the test strip (e.g., a presence or absence of impurities on the test strip), a quality of the test strip (e.g., another quality possessed by the test strip), or the like, or any combination thereof.

### Fabrication

The present electrochemical sensor layer technology can include single-use sensors for non-invasive and painless detection of particular analytes associated with disease or health performance metrics. In some aspects, the disclosed electrochemical sensors can be employed on a device patch that can be fabricated or integrated on various substrates, e.g., including, but not limited to, paper, fabric, bendable and/or stretchable plastics or stretchable elastomeric membranes. In some cases, the electrochemical sensor layer test strip is a single use strip. In some cases, the electrochemical sensor layer test strip is designed for multiple uses. Exemplary electrochemical sensor layer of the present technology can be fabricated using variety of techniques, e.g., including, but not limited to, printing processes such as screen printing, roll-to-roll printing, ink-jet printing, and/or lithographic techniques. The present technology can provide a pain-free single-use non-invasive test as an alternative to single-use strips used for detecting chemicals in blood.

Each layer of the test strip can be deposited on top of the substrate or the other layers via various thin-film and thick-film methods, including electrodeposition, sputtering, physical and chemical vapor deposition, screen printing, transfer printing, dip coating, spray coating, spin coating, doctor blading, inkjet printing, drop casting or microdot patterning. A formed layer can be attached to the previous layers by cold pressing, hot pressing, adhesion, crimping, and embedding. The patterning methods include, but are not limited to, direct deposition or printing, masked deposition or printing, masked etching, laser ablation, laser cutting, mechanical abrasion, or mechanical cutting.

In some aspects, a method of producing an epidermal biosensor includes forming an electrode pattern onto a coated surface of a paper-based substrate to form an electrochemical sensor, the electrode pattern including an electrically conductive material and an electrically insulative material configured in a particular design layout. In some embodiments, there is additionally an adhesive sheet on a surface of the electrochemical sensor layer having the electrode pattern. In some examples, the adhesive sheet is capable of adhering to skin. In some cases, it is structured to include a coating layer on an external surface of the adhesive sheet. In some cases, the paper-based substrate from the electrochemical sensor layer is expose the electrode pattern upon removing the coating layer. In some embodiments of the method, the forming can include performing screen printing, aerosol deposition, or inkjet printing the electrode pattern onto the coated surface of the paper-based substrate.

For example, the electrically conductive material can include a conductive ink, e.g., including, but not limited to, gold, platinum, nickel, copper, silver, and/or silver chloride. For example, the electrically insulative material can include a nonconductive ink, e.g., including, but not limited to, PET, PS, PE, and/or PTFE. In some examples, the electrode pattern can include an electrically semi-conductive material. For example, the electrically semi-conductive material can include a semi-conductive ink, e.g., including, but not limited to, amorphous carbon, carbon black, graphite, carbon nanotubes, and/or graphene. In some embodiments of the method, for example, the electrode pattern can include carbon fiber segments dispersed within the electrically conductive or electrically semi-conductive material.

### Electrode Assembly

Sensors based on electrochemical processes can be used to detect a chemical, substance, a biological substance (e.g., an organism) by using a transducing element to convert a detection event into a signal for processing and/or display. Biosensors can use biological materials as the biologically sensitive component, e.g., such as biomolecules including enzymes, antibodies, nucleic acids, etc., as well as living cells. For example, molecular biosensors can be configured to use specific chemical properties or molecular recognition mechanisms to identify target agents. Biosensors can use the transducer element to transform a signal resulting from the detection of an analyte by the biologically sensitive component into a different signal that can be addressed by optical, electronic or other means. For example, the transduction mechanisms can include physicochemical, electrochemical, optical, piezoelectric, as well as other transduction means.

In some embodiments, a non-invasive electrochemical sensor layer of the disclosed technology includes an anodic and a cathodic electrode contingent. Each contingent includes an electrochemical sensor layer electrode assembly including working electrode, a counter and/or reference electrode. The working, and counter and/or reference electrodes together constitute the electrochemical sensor layer electrode assembly that is utilized for sensitive chemical detection. In embodiments of a two-electrode configuration of the electrochemical sensor, for example, in addition to the working electrode, the second electrode is operable as a reference and counter electrode. In embodiments of a three-electrode configuration of the electrochemical sensor, for example, in addition to the working electrode, one electrode is operable as a reference electrode, and the other electrode is operable as a counter electrode. In some embodiments, for example, the working electrode of the cathode and/or the anode is modified with specific receptors like enzyme, ionophores and/or other reagents for achieving selective detection of the desired chemical analyte. Examples of ionophores include, but are not limited to, sodium ionophores (e.g., nonactin, monensin, etc.), fluoride ionophores (e.g., fluorides, lanthanum fluoride, aluminum fluoride, etc.), chloride ionophores (e.g., tridodecylmethylammonium, quartinary amines, etc.), or the like, or any combination thereof.

In some embodiments, the electrochemical sensor layer is electrically coupled to one or more electrical circuits or electronic devices. For example, the electrochemical sensor layer can include an electrode interface assembly comprising individual or independent electrically conductive conduits disposed on a substrate that are electrically coupled to the anodic and cathodic electrode assemblies. In some cases, the substrate can include bendable and/or stretchable properties. The individual or independent conduits are configured to electrically couple the electrochemical sensor layer electrodes of the anodic and cathodic electrode assemblies to the external electrical circuits or electronic devices, which are able to electrically energize the electrochemical sensing electrodes and to separately electrically energize the electrode for their respective operations.

In some embodiments for alcohol detection, a three-electrode system for the electrochemical sensor layer electrode assembly is preferred. Whereas for glucose detection, the electrochemical sensor layer electrode assembly may include a two-electrode system (e.g., working electrode and counter/reference electrode) because the current measured as a function of glucose concentration is relatively low, such that the two-electrode system is sufficient to detect glucose. For example, in case of alcohol detection, the concentration of alcohol can be relatively high in the biofluid (e.g., sweat), and hence the current measured is higher, and thereby a three-electrode system can be utilized.

In some embodiments, the sensor uses a closely spaced or interdigitated electrode design that ensures a small inter-electrode distance (<1 mm), which enables the ionic pathway between two or more electrodes for signal transduction when in contact with the fingertip. In some embodiments, the first and second electrodes and the first and second current collectors are arranged in the same direction. In some embodiments, the first electrodes may be working electrodes, and the second electrodes may be reference/counter electrodes. the first electrodes (e.g., working electrodes) and the second electrodes (e.g., reference/counter electrodes) are alternately arranged, and adjacent first and second electrodes are spaced apart from each other by a predetermined distance. embodiments, the distance between adjacent first and second electrodes may be about 1 mm. In one embodiment, the distance between adjacent first and second electrodes is smaller than 1 mm. In another embodiment, the distance between adjacent first and second electrodes is larger than 1 mm. In one embodiment, the first electrodes (e.g., working electrodes) and the second electrodes (e.g., reference/counter electrodes) are interdigitated with one another and are arranged radially. In another embodiment, the first electrodes (e.g., working electrodes) and the second electrodes (e.g., reference/counter electrodes) are interdigitated with one another and are arranged parallelly. In some embodiments, the first electrodes (e.g., working electrodes) and the second electrodes (e.g., reference/counter electrodes) are arranged concentrically. In some embodiments, the electrodes in the sensor are radially-aligned. In some embodiments, the interdigitated electrodes are concentric.

In some embodiments, a gel-free sensor includes a plurality of first electrodes extending in a first direction, a plurality of second electrodes extending in the first direction, a first current collector coupled to the plurality of first electrodes, and a second current collector coupled to the plurality of second electrodes. In one example, the first electrodes and the second electrodes are alternately arranged in a second direction. In one example, the first direction is perpendicular to the second direction. In some embodiments, the first current collector is connected to one end of each first electrode, and the second current collector is connected to one end of each second electrode. In some embodiments, the first electrodes may be used as working electrodes, and the second electrodes may be used as reference/counter electrodes. In some embodiments, the first electrodes may include PEDOT:PSS-Prussian blue (PB) cathode, and the second electrodes may include poly(3,4-ethylene dioxythiophene) polystyrene sulfonate (PEDOT:PSS) anode. In some embodiments, the first electrodes and the second electrodes are solid-state interdigitated electrodes (s) printed on a styrene-isoprene-styrene block copolymer (SIS) substrate. In some embodiments, the solid-state interdigitated electrodes (s) are decorated with the glucose oxidase (GOx) enzyme that reacts selectively with the glucose in the fingertip sweat for subsequent detection. In some embodiments, the first electrodes and the second electrodes extend in the same direction (e.g., the second direction) and alternately arranged at a predetermined distance. In one example, the predetermined distance between adjacent first electrodes and second electrodes provide ionic pathways. In some embodiments, the predetermined distance is smaller than 1 mm.

In some aspects, an electrochemical sensor layer includes a substrate formed of an electrically insulative material. In some aspects, a first electrode formed on the substrate of an electrically conductive material, a second electrode configured on the substrate of a material that is electrically conductive and separated from the first electrode by a spacing region, the first and second electrodes capable of sustaining a redox reaction to produce an electrical signal, and a first electrode interface component and second electrode interface component formed on the substrate and electrically coupled to the first electrode and the second electrode, respectively, via electrically conductive conduit, in which, when in contact with the skin and electrically coupled via the first and second electrode interface components to one or more electrical circuits, the device is operable to detect a substance in a local environment of the skin or the wearable item.

In another aspect, a method to fabricate an epidermal electrochemical sensor layer includes depositing an electrically conductive ink on an electrically insulative substrate to form two or more electrodes adjacent to and separated from one another and conduit wires connecting to each of the electrodes, the depositing including printing the ink on a first stencil placed over the substrate, the first stencil including a patterned region configured in a design of the two or more electrodes and the conduit wires to allow transfer of the ink on the substrate, and the first stencil inhibiting transfer of the ink in areas outside the patterned region; curing the electrically conductive ink; depositing an electrically insulative ink on the substrate to form an insulative layer that exposes the two or more electrodes, the depositing including printing the electrically insulative ink on a second stencil placed over the substrate, the second stencil including a printing region configured in a second design to allow transfer of the ink on the substrate, the second stencil inhibiting transfer of the ink in areas outside the printing region; and curing the electrically insulative ink.

Embodiments of the method can optionally include one or more of the following features. In some embodiments, for example, the substrate can include a paper substrate. For example, the paper substrate can include an upper layer and a base paper layer, the upper layer comprising a release agent coated on the base paper layer and structured to peel off to remove the paper substrate. For example, the curing can include implementing at least one of applying heat or ultraviolet radiation to the deposited ink on the substrate. In some embodiments, for example, the method can include forming an electrically semi-conductive layer over at least one of the two or more electrodes by printing an ink of an electrically semi-conductive material on a third stencil placed over the substrate, the third stencil including a printing region configured in a first design of the at least one of the two or more electrodes, the printing region allowing transfer of the ink on the paper substrate, and the third stencil inhibiting transfer of the ink in areas outside the printing region; and curing the electrically semi-conductive ink. In some embodiments, for example, the method can include dispersing carbon fibers in the electrically conductive ink.

### Functionalization and Multiplexed Detection

In some cases, the sensors are designed in a multiplexed manner for detecting multiple biomarkers. In some cases, the multiplexed electrodes for detecting the multiple biomarkers and physiological signals are on the same sensor. In some cases, a sensor device includes a plurality of first electrodes extending in a first direction, a plurality of second electrodes extending in the first direction, a first current collector coupled to the plurality of first electrodes at one end of each first electrode, and a second current collector coupled to the plurality of second electrodes at one end of each second electrode.

In some embodiments, the test strip includes a plurality of electrode arrays, each of which includes the first electrodes and the second electrodes discussed in this patent document. In one example, different electrode arrays 142, 144, 146 can be used to detect different biomarkers Biomarker 1, Biomarker 2, Biomarker 3.

Embodiments of the device can optionally include one or more of the following features. For example, in some embodiments of the device, at least one of the first electrode or the second electrode can include an enzyme catalyst and an electroactive redox mediator, the electroactive redox mediator facilitating the transfer of electrons between the electrode and the active site of the enzyme catalyst configured to sustain a redox reaction. In some embodiments, for example, the device can include an electrically conductive underlayer on the substrate and underneath each of the first electrode and the second electrode, respectively, the underlayer providing separation of the first electrode and the second electrode.

In some embodiments, for example, the working electrode of the cathode and/or the anode is modified with specific receptors like enzyme, ionophores and/or other reagents for achieving selective detection of the desired chemical analyte. The electrochemical sensing layer includes at least an electrochemical transducer with biorecognition functionalities, such as enzymes (glucose oxidase, glucose dehydrogenase, alcohol oxidase, alcohol dehydrogenase, lactate oxidase, lactate dehydrogenase, cholesterol oxidase uricase, urease, ascorbate oxidase, horseradish peroxidase, catalase, tyrosinase, creatinine deiminase, amylase, glutamate oxidase, xanthine oxidase, bilirubin oxidase, hydroxybutyrate dehydrogenase, hydroxybutyrate oxidase, acetoacetate dehydrogenase), ionophores, antibodies, nucleic acids, aptamers, molecularly imprinted polymers, microbes.

The electrode can be functionalized with various chemical/electrochemical transducers, such as enzymes (e.g., lactate oxidase, lactate dehydrogenase, glucose oxidase, glucose dehydrogenase, bilirubin oxidase, uricase, urea oxidase, alcohol oxidase, alcohol dehydrogenase, tyrosinase, catalase), catalysts (e.g., platinum, ruthenium, palladium, rhodium, silver), redox mediators (Prussian blue, Meldola's blue, methylene blue, indigo carmine, 2,2′-bipyridine, 1,4-naphthoquinone, tetrathiafulvalene, tetracyanoquinodimethane, ferrocene) antibodies, ion-selective membranes, silver/silver chloride mixture, molecularly imprinted membranes, or aptamers, for the sensing of biomarkers. In some embodiments, an electrode is functionalized with glucose. In some embodiments, an electrode is functionalized with lactate. In some embodiment, an electrode is functionalized with alcohol.

The glucose sensor of the of the exemplary device includes the anodic and cathodic contingents, in which each contingent includes: an Ag/AgCl electrode that operates as a counter/reference electrode; a printable Prussian-Blue transducer (e.g., which was selected in this exemplary embodiment in view of its high selectivity towards hydrogen peroxide, the detectable product of the GOx enzymatic reaction). In some embodiments, glucose is extracted at the cathodic contingent, and the working electrode of the cathodic contingent (e.g., modified with the GOx enzyme) can operate to selectively detect glucose. In other embodiments where the analyte includes a negative charge, e.g., such as lactate, the analyte is extracted at the anodic contingent, and the working electrode of the anodic contingent can be modified with the agent (e.g., catalyst, such as lactate oxidase (LOx enzyme) in the case of lactate) to selectively detect the analyte. In the exemplary embodiments performed for glucose detection, chitosan was utilized as a polymeric matrix for immobilizing the enzyme on the transducer surface. However, in some embodiments, a different biocompatible polymer other than chitosan can be used. In some instances, while performing reverse iontophoresis, care should be taken to ensure proper contact between the skin and the sensor for efficient glucose extraction and to avoid skin irritation. For example, this can be satisfied by evenly coating a layer of biocompatible agarose gel on each contingent to cover all the electrodes. The resulting glucose sensor can be easily applied to the skin, adhering and conforming to the contours of the epidermis.

In some embodiments, a sensor device may include an additional sensor for measuring resistance and/or temperature. The sensor may include additional closely spaced or interdigitated electrodes for physiological sensing, with physical transducers such as thermoresistive, thermoelectric, piezoresistive, piezocapacitive, piezoelectric, photovoltaic, physisorption, or chemisorption materials that sense physical properties such as temperature, skin moisture level, or pressure.

### Electronic Device

In some cases, a test strip reading device of the present disclosure is an electronic device. In some embodiments, the electronic device is comprised of a circuit board and a mechanical enclosure. In some embodiments, the circuit board includes a microcontroller, a signal generation and processing chip, such as a potentiostat, a data storage chip, and a connector where the test strip can be inserted. In some embodiments, it can include wireless transmission functionality (e.g., Bluetooth, LTE, 5G), antenna, display panels or indicating lights, electrical or mechanical buttons, and a power source such as a battery. In some embodiments, the mechanical enclosure includes the casing to protect the circuit board and the test strip. In some cases, the test strip reading device may have a power lifetime (e.g., a length of time the test strip reading device has operational power supplied to it, a battery lifetime) of at least about 1, 2, 3, 4, 5, 6, 7, 8, 9, 10, 11, 12, 18, 24, or more months. In some cases, the test strip reading device may not be always on (e.g., may be in an off state (e.g., to save power)). In some cases, the test strip reading device is always on (e.g., to provide faster latency in measurement times).

In some embodiments, the circuit board, controlled by the microcontroller unit, can perform various types of electrochemical measurements, including open circuit voltage measurement, chronopotentiometry, chronoamperometry, chronocoulometry, cyclic voltammetry, differential pulse voltammetry, square wave voltammetry, linear sweep voltammetry, alternating current impedance spectrometry, direct current internal resistance measurement.

The circuit board can store the raw signal from the electrochemical measurement and process the data. The raw signal can be converted to the concentration of the target analyte using a pre-programmed algorithm and user-input calibration data. The algorithm may include a two-point or single-point linear regression using the user-input calibration data which is obtained by another biomarker monitoring device, such as a blood glucose monitor or a continuous glucose monitor. Such algorithms can be personalized and individualized per different fingers.

The memory chip can store the number of uses of the test strips, the raw signals, the processed data, the time of measurement, and the personalized and individualized calibration data.

The circuit board can use wired or wireless transmission to deliver the stored information to a secondary device such as a computer or a smartphone or to a cloud-based data center for data analysis and data visualization. A system (e.g., comprising a test strip reading device) can comprise a computer processor operably coupled to the test strip reading device. For example, the computer processor can be contained within the test strip reading device. In another example, the computer processor can be external to the test strip reading device (e.g., in another device local to the test strip reading device, in a cloud computing server, etc.). The computer processor can be configured to use the test strip reading device or data from the test strip reading device to determine a property of the sample (e.g., a blood glucose level, a presence or absence of an analyte, etc.). For example, the computer processor can provide commands to the test strip reading device to perform measurements on the sample. In another example, the computer processor can receive data generated by a test strip reading device and use the data to determine a property of the sample.

The mechanical enclosure can include a moving cover, allowing the test strip to be reachable by a finger by opening the cover. The cover can be closed after use to protect the test strip. The cover can include a replaceable wipe or a squeegee to remove the sweat residue after touching. The mechanical enclosure can include a replaceable wiper so that the user can wipe the finger prior to touching the test strip. The mechanical enclosure can include a mechanical guide to define the exact location of contact by the finger.

In some cases, the test strip reading device may comprise a lid. In some cases, the mechanical enclosure can comprise a lid. The lid can be positioned to protect an uncovered surface of a test strip (e.g., from abrasion, dust, water, unintentional contact, etc.). For example, in a closed position, the lid can cover the test strip, thereby preventing unintentional contact of the test strip. The lid can comprise the same material as the body of the test strip reading device. For example, a polymer test strip reading device can comprise a polymer lid to provide lightweight protection. The lid can comprise a different material as the body of the test strip reading device. For example, a metal lid can be used to provide increased protection against damage to the test strip. The lid may be slidable with respect to the test strip reading device. For example, to expose the test strip, a lid may be slid along the test strip reading device. The lid may be attached to the test strip reading device via a hinge. For example, the lid can be flipped up to expose the test strip. The lid or the test strip reading device may comprise one or more sensors configured to determine an open or closed state of the lid. For example, the lid can comprise a magnet and the test strip reading device can comprise a magnetic sensor to determine the proximity of the lid to the test strip reading device. In this example, when the lid is closed the magnet can be in close proximity to the magnetic sensor, thereby generating a signal that the lid is closed. Examples of sensors include, but are not limited to, positional sensors (e.g., potentiometers, etc.), light sensors, magnetic sensors, electrical sensors, or the like, or any combination thereof. The presence of a lid sensor can enable the system to remind a user to close the lid after use in the case of the user forgetting to, as well as remove false readings occurring while the lid is closed.

The electronic device may comprise optical or electrical sensors to measure various physiological and physical signals from the finger during sweat sensing. The other physiological and physical signals include but are not limited to temperature, moisture level, sweat gland density, sweat rate, transdermal water loss rate, pressure, hydration level, heart rate, and blood oxygen level. The one or more sensors can comprise one or more thermistors, infrared sensors, thermocouples, resistance temperature detectors, moisture meters, capacitive sensors, hygrometers, cameras, piezoelectric sensors, capacitive pressure sensors, electrical sensors (e.g., electrocardiogram sensors), ultrasound sensors, galvanic skin response sensors, impedance sensors, or the like, or any combination thereof.

The electronic device may use such physical or physiological signals in the algorithm to make corrections for the conversion from raw signal to biomarker concentration.

The electronic device may comprise an optical, capacitive, or ultrasonic fingerprint scanner to identify the user and which finger was used for the non-invasive sweat monitoring.

In some embodiments, the disclosed biosensor devices include an integrated electronic backbone for powering the sensor, and signal processing and wireless communication units on the sensor platform, capable of collecting intermittent data from the diabetes patient, and performing large-scale glucose monitoring across diverse patient populations. The exemplary biosensing platform can be readily used for the non-invasive monitoring of other chemical markers present in the interstitial fluid, and for transcutaneous drug delivery.

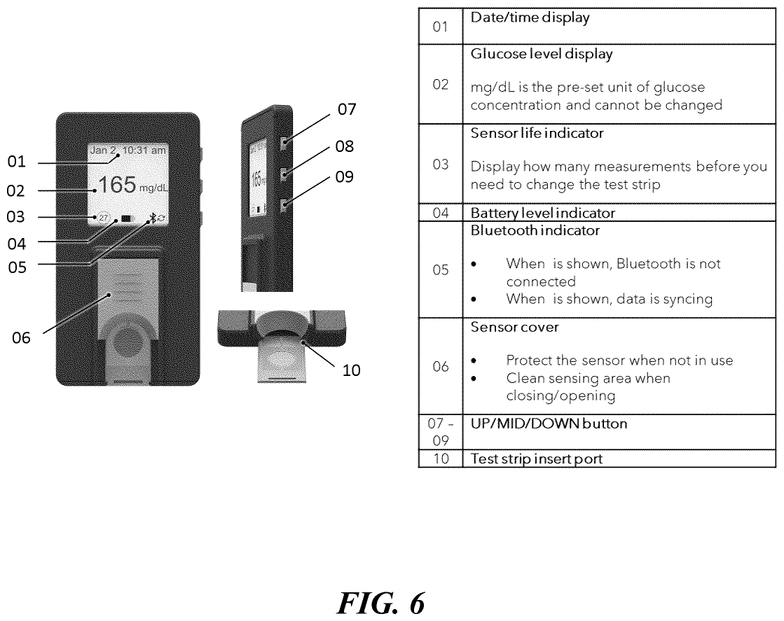

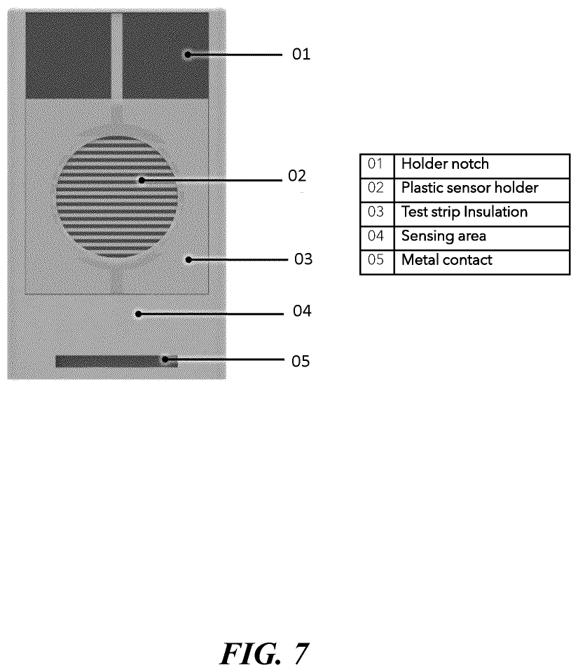

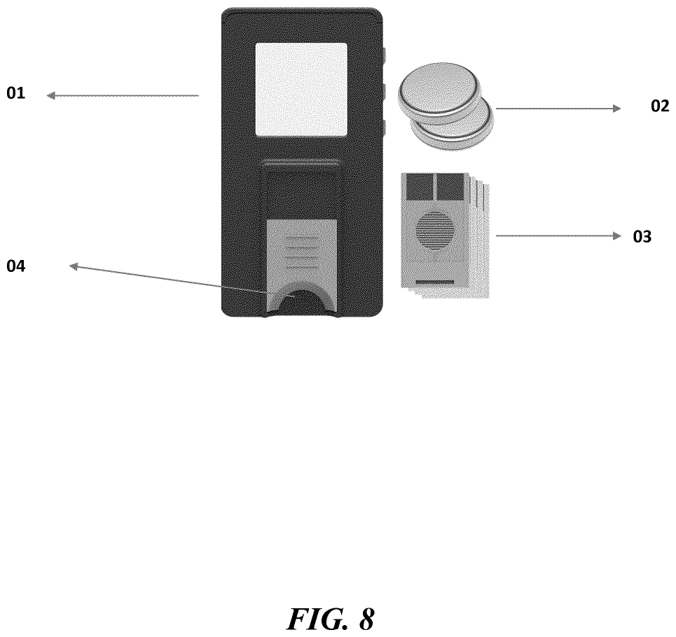

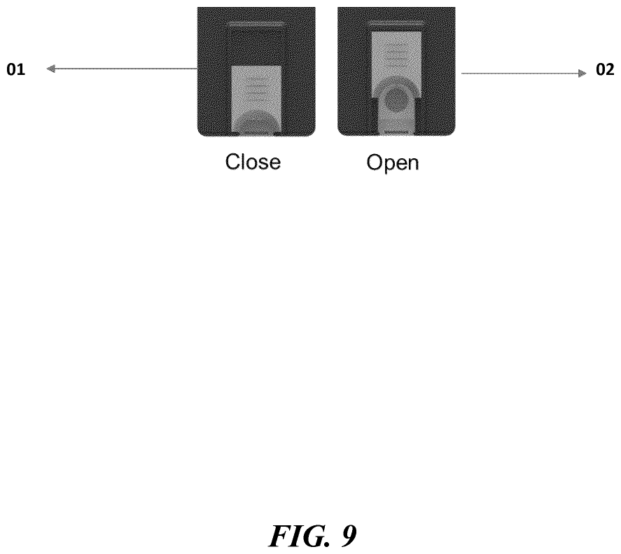

FIG. 6 shows a diagram of an exemplary sensor device of the disclosed technology. FIG. 7 shows a block diagram of an exemplary test strip. As shown in FIG. 8, the sensor device system can be part of a kit that can include a power source 802 (e.g., a battery), a current test strip notch 804, a test strip (sensor signal acquirer) 803, a data processing unit 801 capable of signal processing and communications (e.g., to external devices). The data processing unit can include a processor to process data and a memory in communication with the processor to store data. For example, the processor can include a central processing unit (CPU) or a microcontroller unit (MCU). For example, the memory can include and store processor-executable code, which when executed by the processor, configures the data processing unit to perform various operations, e.g., such as receiving information, commands, and/or data, processing information and data. To support various functions of the data processing unit, the memory can store information and data, such as instructions, software, values, images, and other data processed or referenced by the processor. For example, various types of Random Access Memory (RAM) devices, Read Only Memory (ROM) devices, Flash Memory devices, and other suitable storage media can be used to implement storage functions of the memory unit.

The data processing unit can include an input/output unit (I/O) and/or an output unit that can be connected to, for example, an external interface, source of data storage, or display device. In addition, various types of wired or wireless interfaces compatible with typical data communication standards can be used in communications of the data processing unit via the wireless transmitter/receiver unit 605, e.g., including, but not limited to, Universal Serial Bus (USB), IEEE 1394 (FireWire), Bluetooth, IEEE 802.111, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 2G/3G/4G/LTE/5G cellular communication methods, near field communication (NFC), ultrawideband, and parallel interfaces. For example, a test strip reading device can comprise a USB port (e.g., a USB A port, a USB B port, a USB C port, etc.). The USB port can provide both data transfer as well as power delivery to the test strip reading device. For example, plugging the test strip reading device into a USB port on a computing device (e.g., a computer, a cell phone, etc.) can provide power from the computing device as well as access for a processor of the computing device to the data produced by the test strip reading device. The I/O of the data processing unit can also interface with other external interfaces, sources of data storage, and/or visual or audio display devices, etc. to retrieve and transfer data and information that can be processed by the processor, stored in the memory unit, or exhibited on an output unit of an external device. For example, an external display device can be configured to be in data communication with the data processing unit, e.g., via the I/O, which can include a visual display device, an audio display device, and/or sensory device, e.g., which can include a smartphone, tablet, and/or wearable technology device, among others.

In some cases, the test strip reading device may comprise a display. Examples of displays include, but are not limited to, liquid crystal displays, light emitting diode displays, e-ink displays, or the like, or any combination thereof. The display may be configured to display information to the user about, for example, the number of tests remaining on a test strip, indications of when and for how long to contact the test strip, error messages, results of an analysis performed on the skin of the user, etc. In some cases, the test strip reading device may comprise one or more physical buttons. The one or more physical buttons may permit operation of the test strip reading device without coupling the test strip reading device to an external computer processor. For example, the one or more physical buttons can enable a user to input commands into the test strip reading device to perform an analysis on the user's skin. In some cases, the physical buttons are mechanical buttons, digital buttons, capacitive buttons, or the like, or any combination thereof. In some cases, the test strip reading device may not comprise any physical buttons. In some cases, the test strip reading device may comprise a sensor configured to determine a presence of a user's finger on a test strip in the test strip reading device. For example, a pressure activated switch, pressure sensor, or the like can determine a presence of a user's finger on the test strip. The presence of the user's finger (e.g., determined by the sensor) can start a measurement (e.g., a measurement of a biomarker). For example, a pressure sensor can detect a presence of a user's finer, and the test strip reading device can determine a concentration of a biomarker in the user's sweat. In some cases, the sensor can terminate a reading. For example, when a user removes their finger from the test strip, the sensor can halt a measurement using the test strip. The test strip reading device may comprise a haptic feedback module. For example, the haptic feedback module can provide vibrational feedback when the user activates and deactivates the test strip reading device. The test strip reading device may comprise an audio module. The audio module may comprise one or more audio input devices (e.g., microphones) and/or one or more audio output devices (e.g., speakers). The audio module may be configured to interface with a user to provide, for example, instructions or feedback about the use of the test strip reading device. The audio module may be used to activate or deactivate the device.

In some embodiments, for example, the electronic system can be contained in a housing that electrically connects to the sensor device via electrical contact pads on the substrate of the sensor device that are interconnected to the electrodes of the sensor device (FIG. 6). In such embodiments, the housed electronic system can be a portable device that attaches and detaches from the device (e.g., attaching and detaching the test strip from the holder notch in the device), and be stored on the user to be readily available for the user's next test, e.g., such as in a user's pocket, purse, etc. (FIG. 6). The sensor device can make electrical contact with the portable device via a number of connections including pressure contacts, magnetic contacts, soldering contacts, etc. The housed electronic system can be in wired or wireless connection with a user's mobile communication or computing device, e.g., such as a smartphone, tablet, wearable computing device such as smart glasses, smartwatch, etc., and/or laptop or desktop computer. The exemplary housed electronic system can supply power, operate, and retrieve the acquired physiological-related electrical signals from the sensor device.

Aspects of the present disclosure are directed to wearable technology devices, for example, smartwatches, health-bands, fitness-bands, and smartphones, among others, and combinations thereof, that are enabled to assist their wearers in preventing and/or treating physiological conditions, such as dehydration, stress, fatigue, muscle fatigue, infection, and/or depression by instructing a user when to take a sweat test. Thus, wearable devices include attachable wearable devices that are attached to the body (e.g., wrist, neck, ankle, waist, ear, etc.) as well as carried devices (e.g., mobile phones). Accordingly, various embodiments described herein are adapted to monitor and decide when a person should test their sweat in accordance with the occurrence of one or more certain physiological conditions. Because some physiological conditions can be highly debilitating and even life-threatening, enabling a wearable device to provide such functionality can allow users of this technology to not only quickly recognize certain physiological states, but also, for example, to take remedial measures as directed by the wearable device. Furthermore, by providing a user with an indication of when to take a sweat test eliminates guesswork that may cost the user money. For example, taking a sweat test when prompted may prevent unnecessary sweat tests and costs associated therewith. As described below in detail, such aspects may be facilitated by various GUI's, and other software features running on one or more of a variety of devices, including wearable technology devices (or simply “wearable devices”), and web servers, among other devices. These broad aspects of the present disclosure are described below in connection with a variety of specific examples. That said, those skilled in the art will readily understand that the specific examples described are just that: examples that will inform and instruct those skilled in the art about broad features that they can then implement in a plethora of ways using only routine knowledge and skill in the art.

In some embodiments, the disclosed biosensor devices include an integrated electronic backbone for powering the sensor, and signal processing and wireless communication units on the flexible wearable sensor platform, capable of collecting continuous data from the diabetes patient, and performing large-scale glucose monitoring across diverse patient populations. The exemplary biosensing platform can be readily used for the non-invasive monitoring of other chemical markers present in the interstitial fluid, and for transcutaneous drug delivery.

Embodiments of the subject matter and the functional operations described in this patent document can be implemented in various systems, digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

In some cases, the test strip reading device may comprise a microprocessor. The microprocessor can be configured to interface with the test strip to take a measurement from the test strip. For example, the microprocessor can be electronically coupled to the test strip via one or more leads, and the microprocessor can instruct the sending of electrical signal along the one or more leads. The microprocessor may be configured to receive analog data from the test strip and convert the analog data into digital data. For example, analog electrical measurements from the test strip can be converted to digital values prior to being supplied to a computer processor for processing.

In some cases, a test strip reading device can be configured to be affixed to a computing device. For example, the test strip reading device can have a form factor smaller than a computing device. Examples of computing devices include, but are not limited to, mobile phones, watches, wearable devices (e.g., pedometers, smartwatches, etc.), laptop computers, or the like, or any combination thereof. The test strip reading device can be affixed to the computing device in such a way that the test strip reading device is also operably connected to the computing device. For example, the test strip reading device can be plugged into a physical port of the computing device. In another example, the test strip reading device can be connected via a wireless connection to the computing device it is affixed to. In some cases, the test strip reading device can be reversibly affixed to the computing device. For example, the test strip reading device can be affixed to the computing device using one or more hooks, hook and loop, adhesives, etc. In some cases, the test strip reading device can be permanently affixed to the computing device. For example, the test strip reading device can be integrated into the body of the computing device. In another example, the test strip reading device can be affixed to the computing device using epoxy or the like.

In some cases, the test strip reading device may have a volume of at most about 50, 40, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or fewer cubic centimeters. In some cases, the test strip reading device may have a volume of at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, or more cubic centimeters. The test strip reading device may have a volume in a range as defined by any two of the preceding values. The test strip reading device may be configured to guide a finger or fingertip of a user to the test strip. For example, the test strip reading device may comprise ridges, contours, bumps, etc., oriented so as to aid in the user finding the test strip and positioning their finger properly on the test strip for correct measurement. In some cases, the ridges, contours, bumps, or the combination thereof may be in an outline in a shape of a fingertip. The shape of the test strip reading device may be configured to, when help in the hand of a user, position a finger of the user over the test strip. For example, the test strip reading device may be shaped such that when naturally held in the hand of the user, one of the user's fingers is above a test strip.

The test strip reading device may have a form factor similar to that of a credit card (e.g., narrow, with dimensions similar to a credit card), a keychain (e.g., with a maximum dimension of at most about 5, 4, 3, 2, 1, or fewer inches, configured to be attachable to a keychain), a ring (e.g., configured to be worn on a finger of a user), or the like. The test strip device may be integrated into a mobile computing platform (e.g., a laptop). For example, the laptop can comprise a test strip holder and a lid configured to protect a test strip. The test strip reading device may be configured to be integrated into a mobile telephone case. For example, the mobile telephone case can be configured to hold the mobile telephone as well as a test strip. In this example, the mobile telephone may be communicatively coupled to the test strip reading device. The test strip reading device may be configured to be covered by a portion of the mobile telephone case. For example, a portion of the mobile telephone case may form a lid for the test strip reading device. The test strip reading device may comprise a fixative portion (e.g., an adhesive, a magnet, a hook and loop, etc.) configured to attach the test strip reading device to a mobile telephone case. For example, a test strip reading device can be affixed to a mobile phone case provided by a user. The test strip reading device may be permanently affixed to the mobile phone case. The test strip reading device may not be permanently affixed to the mobile phone case.

### 1. Methods of Detection

Sweat is a non-invasively retrievable biofluid containing rich information of trace-level, health-related biochemical markers. Sweat sensors have shown enormous potential toward monitoring of physiological heath status (e.g., hydration), disease diagnosis and management (e.g., diabetes and gout), and therapeutic drug monitoring (e.g., pain management). However, the presence of skin as a mechanical barrier prevents an uninterrupted access to this information-rich biofluid, and thus a triggering system (i.e., physical exercise, thermal stimulation, or iontophoresis) is necessary to provide continuous access to sweat sample. In contrast to such vigorous active stimulation methods, natural perspiration route has demonstrated immense potential to realize simple, easy, and continuous access to the sweat fluid for chemical analysis. Taking advantage of the high density of eccrine sweat glands (−400 glands cm-2) and the consequent generation of high sweat rates, finger touch-based biosensors have recently been reported for the detection of key sweat biomarkers (e.g., glucose, vitamin C, and cortisol).

In some embodiments, provided herein is a method of non-invasively detecting an analyte from a fingertip, the method comprising: inserting the test strip of any one of preceding claims into the device of any one of preceding claims; placing a portion of skin of a user in contact with the test strip; and obtaining a reading of the analyte from the test strip reading device.

In some embodiments, a method of non-invasively monitoring an analyte from a fingertip, the method comprising: receiving a sample from a skin of a user on the test strip of any one of preceding claims inserted into the device of any one of preceding claims by contact of the skin to the test strip; generating an electrical signal from an electrochemical measurement of the analyte using the solid-state electrochemical sensor; measuring a concentration of the analyte in the sample from the electrical signal; calculating, by the device, a systemic concentration of the analyte in the user from the concentration of the analyte in the sample; and providing the systemic concentration of the analyte on a display of the device.

In some embodiments, the method includes, at 610, obtaining sample of sweat by the device disclosed in this patent document from deposition of the sample of sweat onto the sweat permeation layer of the device from a finger of the individual (FIG. 5), acquiring a plurality of measurements of a level of the analyte using a signal from the sensor disclosed in this patent document (FIG. 7), obtaining, for each of the plurality of measurements of the level of the analyte, a measurement of a concentration of the analyte in blood of the individual, at 602, obtaining an exponential power parameter, an exponential multiplier parameter, and an intercept parameter for a dependence between the obtained measurements of the concentration of the analyte in blood of the individual and the obtained measurements of the level of the analyte in sweat of the individual, and, at 602, using the exponential power parameter, the exponential multiplier parameter, and the intercept parameter to translate a new measurement of the level of the analyte in sweat of the individual to an estimate of the concentration of the analyte in blood of the individual.

An example method for determining a concentration of an analyte in blood of an individual based on some embodiments of the disclosed technology. In some embodiments, the method includes, obtaining sample of sweat by the device disclosed in this patent document from deposition of the sample of sweat onto the sweat permeation layer of the device from a finger of the individual, acquiring a plurality of groups of measurements of a level of the analyte in sweat of the individual using a signal from the device disclosed in this patent document, wherein the sweat is collected by the device from a finger of the individual in contact with the sweat permeation layer of the device, obtaining, for each group of measurements of the level of the analyte in sweat of the individual, a corresponding group of measurements of a concentration of the analyte in blood of the individual, obtaining, for each group of measurements of the level of the analyte in sweat of the individual, values of a linear slope parameter and an intercept parameter for a dependence between the measurements in the group and the measurements in the corresponding group of measurements of the concentration of the analyte in blood of the individual, determining an average value of the linear slope parameter and an average value of the intercept parameter for the groups of measurements of the level of the analyte in sweat of the individual, and, determining a concentration of the analyte in blood of the individual based on the determined average value of the linear slope parameter and the determined average value of the intercept parameter.

### Glucose Testing

In some embodiments, the touch-based non-invasive sweat fingertip glucose detection includes two steps of the sweat collection by touching of a membrane (covering an enzymatic biosensor) and the amperometric detection of the product of the biocatalytic reaction using the biosensor (FIG. 5). The high density of sweat glands in the fingertip ensures sufficient biofluid volume for reliable and reproducible glucose measurements. Sweat collection from the fingertip is performed upon direct contact of the fingertip with the sweat permeation layer (FIG. 5), upon the fingertip touching the sweat permeation layer for a minimal amount of time, e.g., such as for about 1 minute. From the direct contact of the fingertip with the sweat permeation layer, tiny volumes of sweat fluid are taken (collected) in and transfer through the layer, where the collected sweat diffuses to the recognition layer (i.e., the modified and/or unmodified electrodes of the electrochemical sensor) where an enzymatic reaction occurs for detecting a parameter about the analyte in the sweat, which can be processed to determine a parameter of the analyte in blood (discussed elsewhere in this disclosure).

Such painless touch-based glucose sensor represents a promising non-invasive approach to improve diabetes monitoring by increasing the frequency of glucose testing. However, analyzing glucose from sweat is a challenging task. Sweat glucose levels can fluctuate depending on the methodology used for sweat collection. For example, sweat obtained during exercising can underestimate the glucose levels, while iontophoresis can overestimate the glucose levels due to accumulation of glucose on the iontophoretic gels. In addition, contamination from skin components, such as bacteria, body creams and even glucose itself can influence in the measured glucose values. The glucose concertation in sweat ranges from 0.01-1.11 mM, are significantly lower than the blood concentrations (240 mM).

The fingertip touch glucose sensors ensure user-friendly sweat collection as it does not involve exercising or chemical stimulation of the sweat glands. The technology disclosed in this patent document uses ‘personalized’ mathematical approach that improves substantially the sweat-blood glucose correlations and the overall accuracy of glucose testing. Such simple one-time personal calibration accounts for variations in the sweat rate and skin properties among individuals through a distinct sweat-to-blood translation algorithm following a one-time training of the system. The short personal system training involves blood validated sweat signals to estimate the average individual slope (K) and intercept (Io) for each person, for obtaining a personalized sweat-to-blood translation factor. Such initial training and treatment lead to substantially higher Pearson correlation coefficient (Pr) of 0.95, and significantly higher accuracy reflected in an overall mean absolute relative difference (MARD) with 100% of paired points in the A+B region of the Clarke error grid (CEG). These substantial improvements are realized without the need for additional sensors and complex microfluidic network for correcting and normalizing the results. Following such one-time personal training of the system, accurate glucose blood levels can be estimated directly from the individual sweat glucose response over extended periods of several weeks, based solely on his/her sweat signals without the need of blood sampling, by the finger-based detection as depicted in FIG. 5. A single blood calibration is recommended once or twice a month. Such single periodic measurement is analyzed by the software that screens for outliers and updates the existing personal parameters.

Detailed studies demonstrate substantially higher accuracy upon using both the personal intercept and slope compared to using the slope alone. Such greatly improved correlation is achieved even though the values of the slopes and intercept values are substantially different between subjects. In some embodiments, the slope values correspond to the fingertip sweat rate, while the intercepts reflect multiple factors based on the individual skin properties and sweat composition. This simple mathematical treatment can be readily integrated in a software (in, e.g., a hand-held meter or a smartphone app), providing a built-in personal calibration towards autonomous estimate of the sweat-based blood glucose concentration (SG). The methods herein calibrate the subject personal equation based on an initial blood validated sweat response. Once such personalized translation is obtained, blood glucose levels can be directly and reliably estimated from sweat measurements without the need for blood fingerstick validation. A single blood calibration is recommended once or twice a month. Such single periodic measurement is analyzed by the software that screens for outliers and updates the existing personal parameters. By accounting for variability among individuals the new approach provides effective normalization of the sweat glucose response, leading to greatly improved inter person sweat-to-blood correlation parameters, with potential application for the monitoring of other sweat biomarkers.

The electrochemical signal is then converted into blood glucose levels using a personalized algorithm to account for the individual skin properties and sweat rate. Following the successful embodiment of the touch-based sweat-collection/electrochemical detection protocol sensor, the disclosed technology can be implemented in some embodiments to provide a mathematical approach for correlating sweat glucose response to the blood glucose concentrations. Such personalized sweat-to-blood translation algorithm includes measuring the fingertip sweat glucose response and calibrating these current values using the blood glucose levels with a commercial glucometer. Measurements are performed daily at the same time. Sweat and blood glucose levels are measured before and 20 minutes after consuming a meal. An optimized protocol for the finger sweat analysis is strictly followed. First, patients are asked to clean their index finger using a wet tissue and wait for 3 minutes; next, they are asked to touch the sensor for 1 minute. Subsequently, the sweat signal is measured using chronoamperometry at a fixed potential of −0.2V for 60 seconds. Therefore, a mechanical cleaning with water is used due to potential interaction with surfactant residues in soap. This cleaning protocol is followed by an optimal touching time of 1 minute. The calibration plot for each day is analyzed and the average slopes and intercepts are calculated.

### Cortisol Testing

In some embodiments, the disclosed technology can be implemented in some embodiments to provide an effective novel stress-free cortisol sensing platform that allows fast, reliable, and simple detection of cortisol in sweat via a fingertip touch. The disclosed technology can be implemented in some embodiments to leverage such natural sweat sampling method to develop a new stress testing platform, relying on the highly scalable screen-printed electrode modified with a selective MIP recognition layer. Natural perspiration is collected via simple fingertip touch, ensuring that only the endogenous cortisol level is measured, compared to exercise-based contrasting sweat cortisol sensors. To enable the one-step, rapid, reproducible, highly sensitive, and selective cortisol sensing, electropolymerized polypyrrole (PPy) MIP electrodes are synthesized in the presence of cortisol as the template, along with Prussian blue (PB) as the embedded redox probe, hence obviating the need for complex labeling procedure or external redox probes. The subsequent elution of cortisol from the membrane is achieved via overoxidation of PPy, which induces a structural change in the polymer that releases the template cortisol molecule.

This change is confirmed using various surface characterizations and molecular simulations. The template elution resulted in surface recognition cavities that are complementary to the shape and size of the target cortisol molecule. The incorporation of PB within the MIP PPy network leads to a “built-in” electrochemical signaling probe that obviates the need for external redox probes, and hence greatly simplifying the on-body testing compared to common MIP sensors based on such solution-phase redox probe. The resulting user-friendly cortisol sensor, integrating the MIP recognition and the built-in PB-transduction element, thus relies on chronoamperometric measurements (CA) of the PB oxidation current. The selective binding of cortisol within the imprinted cavities, leads to blocking of the PB electron transfer pathways and thus to a decreased PB oxidation current. The extent of such current diminution reflects the sweat cortisol concentration and can thus serve as the analytical signal. Such change in the current is confirmed with cyclic voltammetry (CV) and electrochemical impedance spectroscopy (EIS). The incorporation of the built-in PB redox transducer within the imprinted polymer, and systematic optimization of the experimental parameters, enables fast label-free CA cortisol sensing. In some cases, the test can take only about 3 minutes, which is over ten times faster than common cortisol measurements, thus offers a distinct advantage for capturing sharply fluctuating cortisol levels in response to acute stimulations. Using such fast and simple cortisol testing platform, effortless and stress-free cortisol sensing can be realized toward tracking changing cortisol levels within a diurnal cycle. The variation of cortisol level during physical stimulations, which alters the endogenous cortisol level and is of importance to indicate injury, fatigue, dehydration/malnutrition, can be captured using such sensing platform. The coupling of the simplicity and speed of the touch-based fingertip sweat analysis with a label-free MIP-based electronic detection thus enables dynamic stress-response profiling toward personalized healthcare and the management of personal stress and mental health.

### Dopa Testing

The disclosed technology can be implemented in some embodiments to provide an individualized therapeutic drug monitoring for PD patients, centered on dynamic non-invasive tracking pharmacokinetic profiles of L-Dopa levels in the secreted sweat upon intake of standard pill formulations. Leveraging the natural thermoregulatory sweat sample, a finger-touch L-Dopa biosensor based on some embodiments of the disclosed technology can monitor the dynamic profile of sweat L-Dopa upon intake of standard anti-Parkinsonian medication including L-Dopa-carbidopa (100:25 mg). In some embodiments, the current L-Dopa signal difference is measured in 10-minute intervals shortly after the intake of medication. In some cases, the signal shows a rise in sweat reaching its peak level, after which the signal declined to its background level. In some cases, the signal of the obtained sweat samples versus capillary blood samples showed a similar pharmacokinetic profile with negligible (−10 min) lag time.

The analysis of L-Dopa relies on transfer of the natural sweat to the electrode modified with the immobilized tyrosinase enzyme, upon which sweat L-Dopa is oxidized to dopaquinone via its reaction with the immobilized tyrosinase enzyme. The enzymatically generated dopaquinone is electrochemically reduced back to L-Dopa at the applied potential of −0.3 V, with the resulted amperometric signal correlated with the dynamically fluctuating level of L-Dopa. Such non-invasive, fast, and simple touch-based procedure holds considerable promise toward guiding dose adjustments in PD patients via capturing real-time fluctuations in the sweat L-Dopa levels.

The disclosed method, device, and system can be used to provide a fingertip L-Dopa biosensor. L-Dopa detection may start with (a) touching the sensor with index finger, (b) transfer of natural sweat containing L-Dopa from the skin surface to the electrode surface, where it is electrochemically measured at tyrosinase immobilized electrode, as depicted in FIG. 5.

The disclosed technology can be implemented in some embodiments to provide L-Dopa monitoring using the touch-based sensor. The time course of a cycle of L-Dopa detection in fingertip sweat can include measuring current before touching (2 min), touching (2 min), measurement after touching (2 min), and waiting for the next cycle (4 min). L-Dopa detection is achieved through coupling the tyrosinase enzyme-catalyzed L-Dopa oxidation (catecholase activity) and the subsequent electrochemical reduction of the corresponding quinone product, dopaquinone, at low potentials. The formed reaction cycle not only enhances the sensitivity through amplification of the resulting current signal but also prevents electrode fouling by inhibiting the spontaneous polymerization reactions of the unstable quinone molecules. Tyrosinase enzyme is immobilized on the surface of screen-printed carbon electrodes, followed by crosslinking with glutaraldehyde to prevent leaching of the enzyme.

The performance of the sensor toward following the L-Dopa pharmacokinetics is characterized on healthy patients following the administration of L-Dopa/C-Dopa (100:25 mg) pills which are common oral medication for PD patients. C-Dopa is an amino acid (dopa) decarboxylase enzyme inhibitor and is combined with L-Dopa to enhance the bioavailability of the drug. C-Dopa is an o-diphenolic compound and can be oxidized by the tyrosinase enzyme, and thus may interfere with the target L-Dopa detection. The selectivity of the sensor is challenged via detecting L-Dopa/C-Dopa in 4:1 concentration ratio, similar to the pill composition. In some cases, the selectivity test is performed to detect interference from C-Dopa in the signal from L-Dopa detection. In some cases, the selectivity test indicates minimal interference of C-Dopa as desired for accurate and reliable L-Dopa detection. A

Typical measurements of the target L-Dopa following the pill intake may be carried out at 10 min intervals. The optimal time course of a single 10-min cycle of on-body L-Dopa sensing protocol, including an initial 2-min recording of the background current on the electrode (without fingertip touch) by, followed by placing the index finger on the gel (covering the working electrode) for 2 min, during which sweat diffuses to the electrode surface, and subsequently stepping the potential to −0.3V recording the current signal for 2 min. Following each cycle, the subject is asked to wait for 4 min before starting the next cycle. The L-Dopa current signal starts to increase 10 min after pill intake, reaching its peak maximum at time 30 min, after which signal decreases back to its background level nearly one hour after taking the pill. The touch-based L-Dopa sensor can successfully tracking variation of L-Dopa sweat level. While the blood plasma is the ‘gold standard’ matrix for therapeutic monitoring of L-Dopa, this analysis method relies on LC-MS centralized instruments. To further confirm the reliability of the developed protocol based on touch-based sweat L-Dopa detection, the feasibility of data validation between sweat and blood samples is investigated to confirm that the peak-shaped temporal profile using sweat matches closely the corresponding blood L-Dopa concentration (with a short −10-min time delay).

In certain embodiments, the disclosed technology is implemented in some embodiments to simultaneously provide drug detection methods and devices. Driving under the influence of illicit or licit drugs such as cannabis and alcohol represents one of the major safety concerns due to the strong synergistic effect of these substances. Therefore, a rapid in-situ testing of such substances is needed to decrease the risks of road accidents. Thus, the disclosed technology can contribute to the accurate and fast decentralized, detection of drugs using finger sweat sensor combined with the mathematical approach. The disclosed technology can be used as a personal safety system for car ignition where the finger sweat sensor is directly integrated to the car's ignition, including but not limited to the on/off button, the car's keys, etc. Multiple sweat drug molecules can be detected simultaneously for drug screening and identification. The software used for personalized quantification of such drugs can include a drug data base for identifying the substance in sweat. The disclosed technology can promote such important and needed application for self-monitoring towards safety, besides enabling law enforcement personnel to screen drivers during traffic stop, addressing the growing concerns of drug-impaired driving.

In some cases, a method can comprise providing a test strip reading device holding a test strip (e.g., a test strip as described elsewhere herein). For example, the test strip may comprise a solid-state electrochemical sensor layer and a conductive layer. The test strip may be contacted with a portion of a subject's skin. The solid state electrochemical sensor layer and the conductive layer may be used to generate an electrical measurement based on a biomarker from the portion of the subject's skin. The electrical measurement may be used to determine a property of the biomarker of the subject.

The property of the biomarker may be as described elsewhere herein (e.g., a blood glucose level of the subject). For example, the method may be a method of non-invasively determining a blood glucose level of a subject. The determining of the property of the biomarker may be performed in an absence of pre-calibration or normalization. For example, the raw output of the test strip reading device may be used to determine the property of the biomarker without a pre-calibration being applied. In some cases, the test strip reading device has been calibrated a single time for a plurality of tests. For example, a given individual determination of a property of the biomarker may not comprise calibration, but an overall calibration can be performed for the test strip reading device. In this way, the individual determinations may be faster while maintaining accuracy. In some cases, a calibration measurement can be used in the determining of the property of the biomarker. For example, the test strip reading unit can be calibrated.

In some cases, additional sensors such as those described elsewhere herein can be used to provide additional data points in the measurement of the biomarker. For example, a skin roughness or moisture content measurement may be used to aid in accurately determining the property of the biomarker. In this example, the moisture content can be used to normalize the reading of the biomarker for different conditions on the finger of a user. In some cases, the use of the additional sensors of the present disclosure can enable calibration free measurement of the property of the biomarker. For example, using a combination of sensor data, the property of the biomarker can be determined without a separate calibration profile for the biomarker for the user. In this way, a test strip reading device of the present disclosure can be used on a subject without prior calibration which can be useful in, for example, emergency situations where a prior calibration may not be available.

In some cases, prior to contacting the test strip, a lid affixed to the test strip reading device can be opened to expose the test strip. For example, the user can open the lid to access the test strip. In some cases, the test strip is reusable as described elsewhere herein (e.g., for at least about 15 times).

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein the term “about” refers to an amount that is near the stated amount by 10% or less.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The section headings used herein are for organizational purposes only and are not to be construed as limiting the subject matter described.

### EXAMPLES

The following examples are included for illustrative purposes only and are not intended to limit the scope of the disclosure.

### Example 1: Touch-Based Sweat Glucose Monitoring System

The Touch-based Sweat Glucose Monitoring System is used for the quantitative measurement of glucose (sugar) in fresh natural perspiration from the fingertip and convert it to corresponding blood glucose levels upon personalized calibration. The disclosed technology can be implemented in some embodiments to provide a unique solid-state interdigitated electrode transducer for direct touch-based glucose monitoring without the need for any sweat-extraction mechanisms. Unlike early studies based on stimulated sweating via exercise or iontophoresis, which reported a low correlation to blood glucose, the present use of natural perspiration offers high accuracy in predicting blood glucose levels in connection to a single first-day personalized calibration. With the attractive features of the new user-friendly non-invasive biosensing method and its high-quality data, the disclosed technology can be implemented in some embodiments to enable non-invasive glucose monitoring that can become an important component of diabetic self-care.

FIG. 6 shows an example sensor device for measuring a biomarker in a biofluid (e.g., glucose in sweat) based on some embodiments of the disclosed technology. The components of the sensor device are given in Table 1. In some embodiments, the method includes placing a sensor system in contact with a skin of a subject, measuring a biomarker in a biofluid (e.g., sweat) from the skin of the subject using the sensor system as shown in FIG. 5. The sensor system may be composed of the Sweat Glucose Meter as shown in FIG. 6 and Test Strips as shown in FIG. 7. The test strip components are shown in Table 2. The system operates on the principle that glucose in the capillary blood can diffuse through the sweat glands and discharge with the natural perspiration from the fingertips. The strip has a holder notch for the user to hold the strip without touching the sensor 701. The plastic sensor holder is depicted in 702. The test strip is insulated as shown in 703. The sensing occurs in the sensing area 704. The Test Strip is functionalized with enzyme GOx and a small electric current can be detectable upon reacting to the glucose in the natural perspiration. The strength of this current can be correlated with the sweat glucose level upon an initial two-point calibration. The Touch-based Sweat Glucose Sensing System displays 602 the sweat glucose level and then stores it in the system memory. In some embodiments, the Glucose Sensing System is part of a kit that contains the Touch Based Sweat glucose Monitor 801 with an notch for inserting the test strips 804, 4 sensors 803 and pre-installed batteries 802. In some cases, the kit further contains a manual with user instructions.

TABLE 1

| | |
|---|---|
| 01 | Date/time display |
| 02 | Glucose level display |
| | mg/dL is the pre-set unit of glucose concentration |
| | and cannot be changed |
| 03 | Sensor life indicator |
| | Display how many measurements before you need to |
| | change the test strip |
| 04 | Battery level indicator |
| 05 | Bluetooth indicator |
| | When is shown, Bluetooth is not connected |
| | When is shown, data is syncing |
| 06 | Sensor cover |
| | Protect the sensor when not in use |
| | Clean sensing area when closing/opening |
| 07-09 | UP/MID/DOWN button |
| 10 | Test strip insert port |

TABLE 2

| | |
|---|---|
| 01 | Holder notch |
| 02 | Plastic sensor holder |
| 03 | Test strip Insulation |
| 04 | Sensing area |
| 05 | Metal contact |

In some embodiments, the touch-based sensing system comprises a display monitor 601-605. The display monitor is designed to display information regarding the level of analyte as measured in the biofluid 602. The display monitor has other indicators such as sensor life indicator 603. In other cases, the display monitor displays information regarding battery life 604, or regarding connection to Bluetooth sources 605, or date/time 601.

The sensor system comprises a meter that is turned on by the user, by pressing a key until the Welcome screen appears. In other cases, the system comprises a meter that is turned on by sliding the sensor cover to the testing position 902 from the idle position 901. The sensor meter comprises an up/down button 607, 609 to change the number and MID button 608 to jump to the next digit/next step. At the end of the setting, the user presses MID button 608 to confirm if the time and the date is correct, or press DWN button 609 to redo the setting.

The device prompts the user to set up a personalized calibration profile before using the meter for the first time, which requires two sets of glucose measurements at two different glucose levels—one at fasting and one hour after meal. For each set of measurement, the user performs one fingerstick measurement using a commercial grade blood glucose meter and one measurement with the Touch-based Sweat Glucose Monitor System. The sensor meter is designed to be personally calibrated to each person, and to a specific finger of the person. The sensor meter is designed for use with the same finger that was used for personal calibration of the Glucose Monitor System measurement. The sensor meter is designed to measure glucose values between 40-400 mg/dL and the user must calibrate with two calibration entries that have a different above 20 mg/dL.

Before taking a fingerstick test, the user washed their hands and used glucose test strips that are unexpired and that have been stored properly. After performing the glucose measurement test as per instructions, the user inputs the result into the system touch-based system calibration profile accurately.

In order to complete the calibration profile, the user measures the glucose level on the touch-based sensor by pressing the index finger on the sensor until the screen indicates completion, before pressing the MID button to confirm. The second set of measurements are taken when the glucose level has changed significantly (>20 mg/dL) and once again, the user measures the glucose level on the touch-based sensor by pressing the index finger on the sensor until the screen indicates completion, before pressing the MID button to confirm.

The sensor meter requires that the selected index finger of the user is clean and dry before use. The sensor meter has a cover that the user pushes to the open position using the selected index finger and then presses on the sensing area to begin a test. The user hears a click when pressing with sufficient pressure. The sensor meter has a cover that is closed by the user after measurement. The results are displayed on the screen and stored in the meter's memory. If the glucose test results are lower than 20 mg/dL, the sensor meter indicates to the user that this may indicate hypoglycemia and is stored in the mobile app. Glucose test result above 600 mg/dL is indicated as possible hyperglycemia (high blood glucose) or that the result is above our measuring range. The high result is stored in the meter. On the mobile app it will be saved as a test result of >600 mg/dL.

In some cases, the test strip can be used for 30 times. After each measurement, the number of usage left is displayed on the left bottom corner of the screen. The sensor meter indicates to the user that a replacement of the test strip is required after the test strip has been used for 30 times. A change sensor notification will be shown if the sensor has been used for >30 times. 603 To replace your sensor, the user pulls out the test strip using the holder notch. 610 The user inserts a new test strip into the test strip insert port and pushes until it is fully inserted. If not fully inserted, the screen will show the insert strip notification until a new sensor has been detected.

In some embodiments, the electronic system can include a display to present the analyzed data to the user. The sensor system stores 200 glucose measurement results. The non-invasive electrochemical sensor layer is connected to an external electronic system, which can be portable and/or wearable on the user. The electronic system is used to power the electrochemical sensor layer and analyze the acquired sensor signal (e.g., detected electrical current, voltage, etc.) to produce data on the analyte, e.g., such as the chemical concentration. The information can be transmitted wirelessly from the electronic system to a user computing device, e.g., such as a smartphone, tablet, wearable computing device such as smartglasses, smartwatch, etc., and/or laptop or desktop computer. The electronic system can be connected to such user computing devices via physical contact (e.g., wired) or wirelessly using RF or Bluetooth communication, or other wireless communication techniques. Bluetooth technology lets the users wirelessly send their readings to a mobile device where they can graph their results.

### Example 2: Fabrication of the Sensor

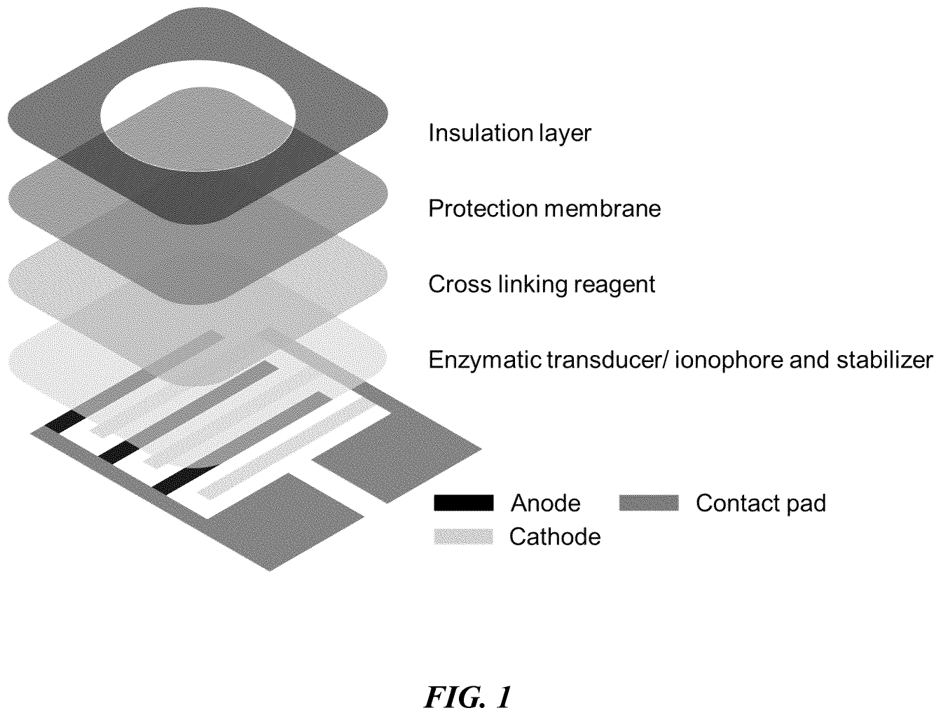

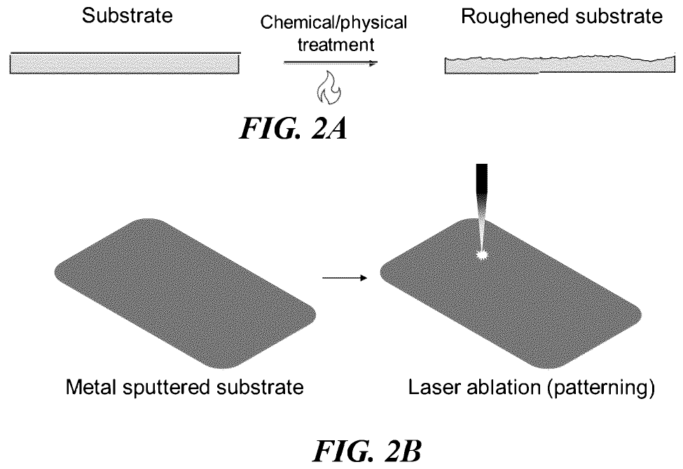

FIG. 1 shows exploded-view schematics of the structure of the sensor. The present disclosure can be implemented in some embodiments to provide a solid-state touch sensor that enables a frequent, accurate, non-invasive glucose monitoring. The sensor has a substrate that is subjected to chemical and mechanical treatment procedures as shown in FIG. 2A. In some cases, the substrate is fabricated using lithography/patterning techniques. FIG. 2B.

The electrochemical sensor layer on the test strip can be fabricated using layer-by-layer screen-printing. For example, the sensor in one instance is customized with multiple inks. The sensor, in one instance, is overlaid first with the electrochromic poly(3,4-ethylenedioxythiophene) polystyrene sulfonate (PEDOT:PSS) ink, next silver ink for interconnection, and next, an insulating resin composed of SEBS. The PEDOT:PSS ink is formed using a mixture of ink prepared using 1 g of PEDOT:PSS paste, 0.2 mL of toluene, 0.15 mL DBSS (75 mg/ml in DI water), and 0.0135 mL of fluorosurfactant FS-65. To make the PEDOT:PSS-PB ink, similar components may be used, with additional 20 mg of PB powder that is added per 1 g of mixture. The stretchable silver ink is synthesized, in one instance, using a combination of silver flakes, toluene, and SEBS mixed in a weight ratio of 4:2.37:0.63. The inks are homogenized by mixing them in a dual asymmetric centrifugal mixer, e.g., with a speed of 1900 RPM for 5 min. The insulating resin is prepared by dissolving SEBS in a toluene solution (4:10 weight ratio). The solution is then mixed at 1900 RPM for 20 min or until the SEBS is completely dissolved in the solution.

After ink preparation, the flexible substrate for printing the fingerprint sensor is fabricated. The surface of the substrate can be mechanically, chemically, or physically treated to change its surface roughness, hydrophobicity, dielectric constant, and electrostatic charge for improved adhesion and compatibility with sweat sensing. The treatment process includes one of acid or base chemical etching, plasma etching, electroplating, abrasion, laser ablation, sputtering, heating, and physical or chemical vapor deposition. For example, a polyethylene terephthalate substrate can be soaked in a 1M HCl bath at 80° C. for 1 hour for acid etching to improve its hydrophobicity. In some cases, the substrate can thereafter be rinsed with water and dried to remove access chemicals. In some cases, the substrate may undergo ozone plasma treatment for 20 minutes to remove chemical contaminants and enhance its adhesion with subsequent deposited layers. The substrate is comprised of one or several types of plastics, ceramics, or natural materials, such as polyethylene, polypropylene, polyethylene terephthalate, polyester, polyimide, polydimethylsiloxane, alumina, silicon, and paper.

In some embodiments, a SIS layer of 1000 μm is formed on top of a plastic sheet of PET. The thin layer is dried at 60° C. for 30 min. The printed electrode patterns (e.g., area of electrodes 0.02 cm²) are designed in software and are transferred to stainless steel plates (e.g., 12×12 in²) etched to fabricate metallic stencils. The electrochemical system, composed of a working and reference electrodes, is screen printed using an MPM-SPM semiautomatic screen printer. The printing process consists of printing first the reference electrode using the PEDOT:PSS ink on the SIS substrate followed by a curing step of 30 min at 120° C. Next, the working electrode is printed using the PEDOT:PSS-PB ink, similar drying conditions are applied after printing the electrode. Subsequently, the silver interconnection pattern was printed on top of the electrode system followed by a curing step of 15 min at 90° C. The interconnections are insulated using the SEBS resin. Finally, the insulator is allowed to dry for 10 min at 90° C. to obtain the printed electrode. The electrodes can then be modified by drop-casting a mixture 6 μL of glucose oxidase (20 mg/mL in PBS, 0.1 M, pH 7.3) and 3 μL of glutaraldehyde (1% in DI water) on top of the exposed electrodes surfaces. After modification, the electrodes are stored overnight at 4° C. inside a refrigerator.

In one instance, the electrochemical sensing layer can comprise a combination of cofactors, mediators, stabilizers, polymers, or plasticizers. For example, in some embodiments: graphite, toluene, acetone, ethanol, glutaraldehyde, glucose, glucose oxidase (GOx), Ag flake, potassium chloride (KCl), sodium chloride (NaCl), sodium phosphate anhydrous, Prussian blue, and sodium dodecylbenzene sulfonate (DBSS) is used for fabrication of the interdigitated electrode based on some embodiments of the disclosed technology. Styrene-ethylene-butylene-styrene (e.g., SEBS G1645) triblock copolymer is used for fabrication of the interdigitated electrode based on some embodiments of the disclosed technology. In one instance, the screen printable PEDOT:PSS paste is used for fabrication of the interdigitated electrode based on some embodiments of the disclosed technology.

In addition to the electrochemical sensing layer, the sensor comprises an insulation later, counter interference layers, a protective permeable layer, and a conditioning layer. Each layer of the test strip is deposited on top of the substrate or the other layers via various thin-film and thick-film methods, including electrodeposition, sputtering, physical and chemical vapor deposition, screen printing, transfer printing, dip coating, spray coating, spin coating, doctor blading, inkjet printing, drop casting or microdot patterning. A formed layer is attached to the previous layers by cold pressing, hot pressing, adhesion, crimping, and embedding. Each layer of the test strip is patterned separately or together into a desired shape for optimal sweat contact and signal transduction.

### Example 3: In-Vitro Sensor Characterization

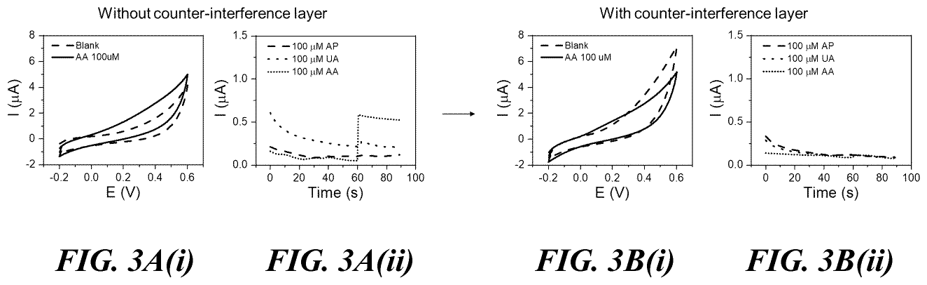

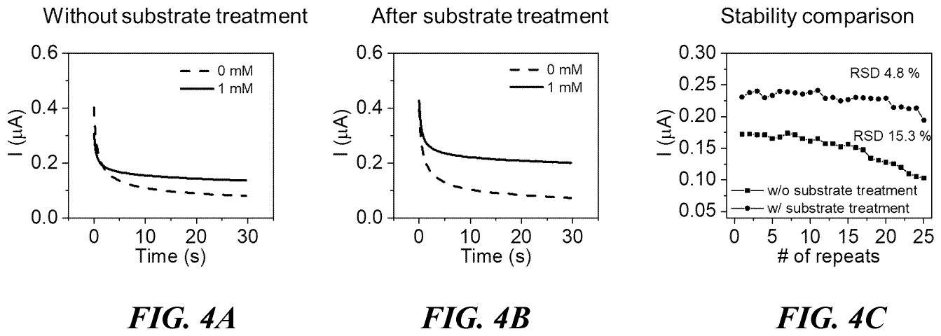

The in-vitro characterization of the touch-based glucose sensor was conducted using both 0.1 M PBS (pH 7.3) and ascorbic acid (100 μM). The in-vitro characterization of the touch-based glucose sensor included characterization of the sensor's selectivity and reproducibility (FIGS. 3-4). FIG. 3A(i) shows characterization of the sensor without counter interference layer in the presence and absence of Ascorbic acid including (i) 2-electrode CV, (ii) CA at different potentials from −0.2 V to 0.6 V. FIG. 3B (i) shows characterization of the sensor in the presence and absence of ascorbic acid with a counter interference layer including (i) 2-electrode CV, (ii) CA at different potentials from −0.2 V to 0.6 V.

Additionally, the selectivity test was performed in PBS and by spiking 100 μM of ascorbic acid (AA), acetaminophen (AP), and uric acid (UA) in the absence of a counter interference layer (FIG. 3A (ii)) and in the presence of a counter interference layer (FIG. 3B (ii)). FIG. 3A (ii) shows selectivity of a sensor: CA response of the sensor in PBS (blank) and following addition of 1 mM glucose before substrate treatment. FIG. 3B (ii) shows selectivity of a sensor: CA response of the sensor in PBS (blank) and following addition of 1 mM glucose after substrate treatment.

Highly reproducible CA signals are observed for these repetitive touch-based measurements, leading to RSD values of 4.8% with substrate treatment procedures (FIG. 4B) as compared to 15.3%, respectively without substrate treatment (FIG. 4A). The highly reproducible current signals, with a low RSD value of 3.9% (n=20), reveal the good repeatability for multiple repeated frequent measurements (FIG. 4C).

### Example 4: Cortisol Monitoring and Validation in Circadian Cycles

The disclosed technology can be implemented in some embodiments to provide sweat biomarker monitoring methods and devices. The personalized processing of touch-based fingertip sweat assays offers simplified accurate tracking of a combination of key sweat biomarkers, such as levodopa, cortisol, alcohol, lactate, ketone bodies, or uric acid as well as illicit drugs or tetrahydrocannabinol (THC). For example, tracking cortisol level fluctuations is important in understanding the body's endocrine response to stress stimuli. Traditional cortisol sensing relies on centralized laboratory settings, while wearable cortisol sensors are limited to slow and complex assays. The disclosed technology can be implemented in some embodiments to provide a simple touch-based sensor for rapid cortisol detection. In one embodiment of the technology, the entire assay can take around 3 min which is extremely important for capturing instantaneous and sharp variations in cortisol levels. The sensor readily samples natural sweat from the fingertips onto the cortisol-imprinted polypyrrole, with embedded Prussian blue redox probes. FIG. 5 is an illustration of an experimental setup for visualizing the perspiration on the fingertip when pressed against the sensor. stressful and lengthy sweat-extraction procedures. Correlating values of sweat biomarkers with the corresponding blood values is current a challenge for the sweat sensor industry, the new methodology disclosed here makes possible to account for the inter-individual variability for accurate estimate of the blood concentration.

The performance of the new cortisol sensor is first evaluated by monitoring the variations of endogenous cortisol levels during the diurnal cycles. Studies have shown the correlation of cortisol levels with the circadian rhythm, where larger cortisol concentrations are present during the morning, decreasing during the day, and finally reaching lower levels in the evening. Dynamic tracking of such cortisol levels semi-continuously is of considerable importance for assessing the chronic stress level of individuals. Daily variations in the response of the touch-based sweat cortisol sensor are thus monitored and validated. The cortisol levels of 5 patients are measured at 7 a.m. and 5 p.m. on the same day using fingertip sweat, along with validation via immunoassay of pilocarpine-stimulated sweat samples.

As an example, the optimized touching and incubation times are used, and the cortisol signal is acquired with a portable device with a test strip sensor modified for cortisol detection. Considerable differences, ranging from 86 to 200×10⁻⁹ m cortisol, are observed for subjects using the finger-based cortisol sensor and amperometric cortisol response of three patients for the morning/evening experiment show considerable variance. The background signal is measured with the sensor surface, followed by the sweat cortisol measurements in the morning (red curve) and the evening (blue curve). In some instances, a new sensor is used for recording each response. The data from the sensor may be correlated (with Pearson's r=0.96) between the cortisol sweat concentrations estimated by the fingertip MIP sensor and by the corresponding immunoassay. Following the sweat validation testing, the new sweat finger sensor is successfully used to monitor the morning and evening cortisol levels of additional patients, displaying clear differences in the concentrations during these periods. The fast and convenient use of the finger cortisol sensor is demonstrated by monitoring the cortisol levels of several patients throughout the day. For this, the sensor response is recorded every 2 hours, over 12 hours, from 7 a.m. to 7 p.m. A gradual decrease in the sweat cortisol level is observed for all patients between the morning to the evening measurements.

Attempting sweat stimulation during physical activity would result in a mixture of chemically and exercise-induced sweat, which clearly shows the need for having effortless sweat cortisol sensing, since exercising inducing cortisol can affect the endogenous cortisol levels. Patients who include an exercising routine (involving 30 min indoor biking) in their daily activities during such semi-continuous cortisol monitoring, show increased sweat cortisol levels right after exercising, which decreased to the endogenous levels within 2 h. Therefore, in order to assess and validate the exercise stress stimuli, the sweat induced protocol is used right after the exercise routine.

### Example 5: Multiplexed Detection of Biomarkers

In some embodiments, the applications of the data processing methodology can be combined with several biosensors, including but not limited to levodopa biosensor, modified via tyrosinase enzyme or non-enzymatic sensor via voltametric techniques, lactate biosensor modified via lactate oxidase enzyme (or other recognition elements), cortisol biosensor modified via molecularly imprinted polymerization (MIP) (or other recognition elements), ketones bodies biosensors using P-Hydroxybutyrate dehydrogenase enzyme modified sensors (or other recognition elements), glucose biosensor using glucose oxidase enzymes (or other recognition elements), THC sensors using either nanoparticle, CNTs or MIP modified sensors (or other recognition elements), illicit drugs such as cocaine using bare carbon electrode (or other recognition elements), and alcohol using the enzyme alcohol oxidase (or other recognition elements).

For example, in addition to detecting cortisol as described in the previous example, the disclosed technology can be implemented in some embodiments to provide a data processing approach for correlating sweat analyte response of ketone biomarkers in natural passive perspiration and their blood concentrations. A P-Hydroxybutyrate dehydrogenase enzyme modified sensors biosensor is used for measuring sweat ketones. After contacting the skin for a determined amount of time, the collected sweat reaches the recognition layer, where the analyte is measured. As discussed above, the working electrode of a screen printed 3-electrode system is modified with the enzyme for detecting ketones and cortisol. Sweat is collected from the fingertip during, e.g., 1-minute touching after proper washing of the hands. After collection, sweat ketone signal is obtained by chronoamperometry. The signal is obtained twice a day for one week.

After data acquisition the personalized correlation equation can be determined. For this, data is acquired for several days and validated with appropriate approaches. For example, the determination of sweat ketones can be validated using commercial urinealysis. Urine sample is collected and analyzed prior each measurement for the validation steps. After data collection, the linear slope and intercept obtained each day is averaged and a personalized universal equation is derivate for direct conversion of the signal intensity to the blood concentration. As discussed above, a linear correlation between the two points (sweat and urine ketone) is obtained for each day of analysis and an averaged slope and intercept is calculated for the user. These personalized values account for the individual sweat parameters such as sweat rate and composition. The personalized general equation is then used to direct translate the sensor signal into blood ketone values in addition to cortisol levels (obtained as described in earlier examples). Therefore, various embodiments of features of the disclosed technology can be made based on the above disclosure, including the examples and embodiments listed above.

### Example 6: Test Strip Reading Devices

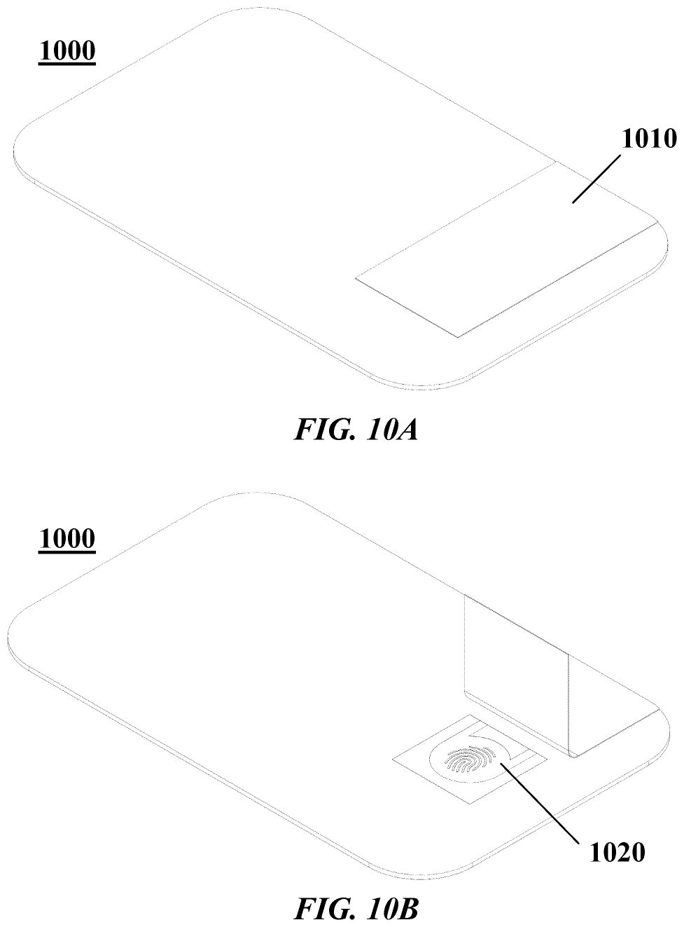

FIGS. 10A-10B show examples of a credit card form factor test strip reading device 1000. The credit card form factor test strip reading device can comprise a lid 1010 covering a test strip 1020. The credit card form factor test strip reading device may be disposable. For example, the credit card form factor test strip reading device may not be configured with a removable test strip. The credit card form factor test strip reading device may comprise a wireless power delivery or wireless data transfer modules as described elsewhere herein. For example, the credit card form factor test strip reading device may not comprise ports or other interface points. The credit card form factor test strip reading device can provide a low cost and compact test strip reading device.

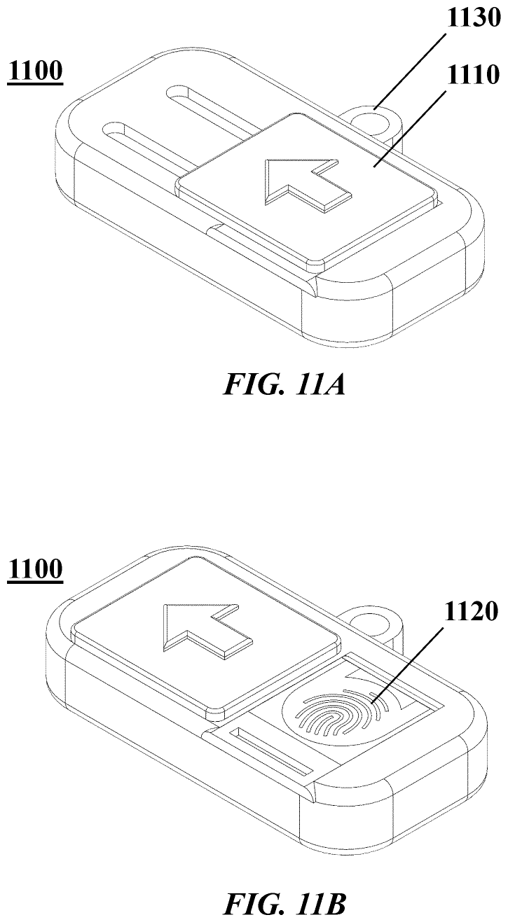

FIGS. 11A-11C show examples of a key chain form factor test strip reading device 1100. The lid 1110 can be positioned to protect the test strip 1120 and be movable to expose the test strip. Loop 1130 can be configured to interface with a chain, strap, or the like, thereby providing the ability to secure the key chain for factor test strip reading device. In some cases, the test strip can be removed 1140 from slot 1150 and replaced with a new test strip. In this way, the key chain for factor test strip reading device can be reusable for a plurality of test strips. As described elsewhere herein, the key chain for factor test strip reading device can comprise a battery configured to power the key chain for factor test strip reading device. In some cases, the key chain for factor test strip reading device may be disposed of once the battery is no longer operational (e.g., the battery may not be replicable).

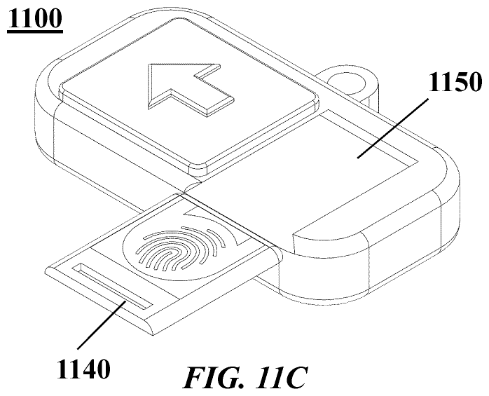

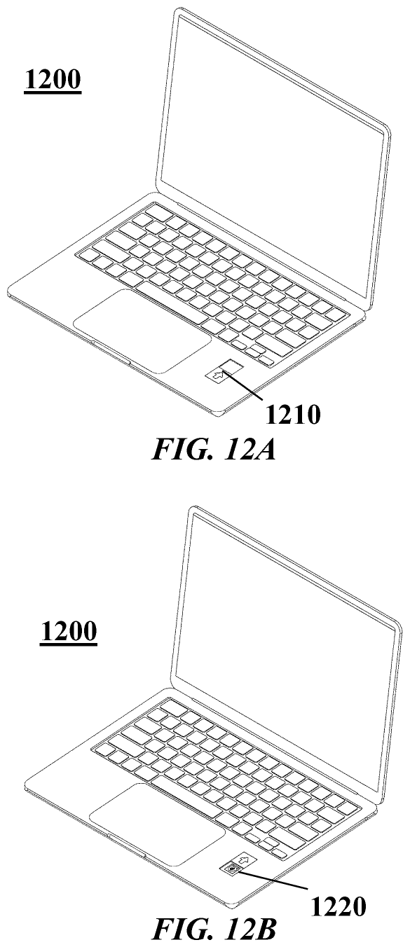

FIGS. 12A-12B show examples of a laptop form factor test strip reading device 1200. The laptop form factor test strip reading device may comprise a lid 1210 configured to cover a test strip 1220. The laptop form factor test strip reading device may be integrated into a laptop computer (e.g., a personal computer, an Apple® laptop, a Chromebook®, or the like), and the computer processor in the laptop can provide the computing power for the laptop form factor test strip reading device. Similarly, a battery within the laptop can provide the power to the laptop form factor test strip reading device.

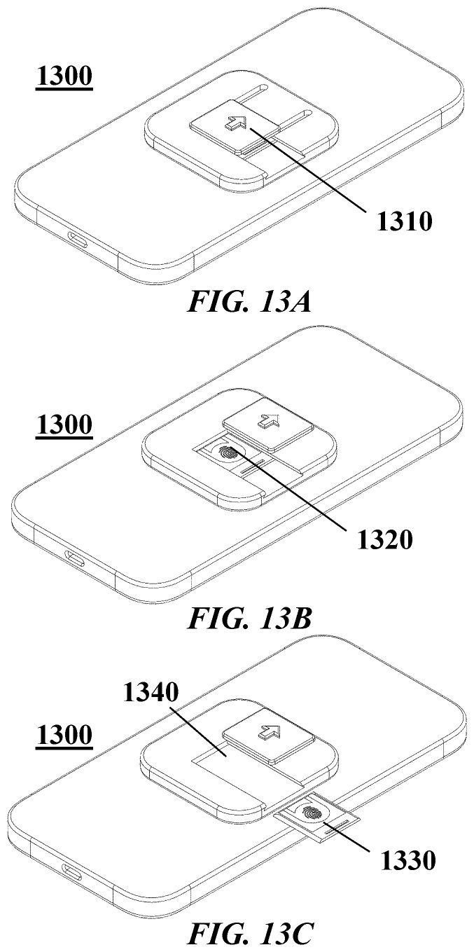

FIGS. 13A-13C show examples of a mobile phone case form factor test strip reading device 1300. The phone case can be configured to hold a cellular telephone. In some cases, the phone case form factor test strip reading device can be configured to be affixed (e.g., with adhesive) directly to the mobile phone. The phone case form factor test strip reading device can comprise a lid 1310 configured to cover a test strip 1320. The test strip may be removable 1330 from slot 1340. In some cases, the phone case form factor test strip reading device can be wirelessly powered by the mobile phone. In some cases, the phone case form factor test strip reading device can wirelessly communicate (e.g., via Bluetooth®) with the mobile phone.

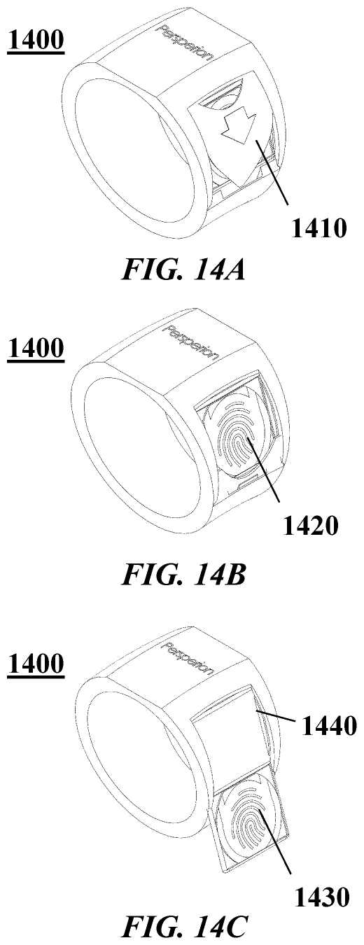

FIGS. 14A-14C show examples of a ring form factor test strip reading device 1400. The ring form factor test strip reading device may comprise a lid 1410 configured to cover a test strip 1420. The lid may be removable to expose the test strip. In some cases, the lid is slidable to expose the test strip. In some cases, the test strip is removable 1430 from slot 1440, and the ring form factor test strip reading device may be reusable. In some cases, the ring form factor test strip reading device may comprise a rechargeable battery.

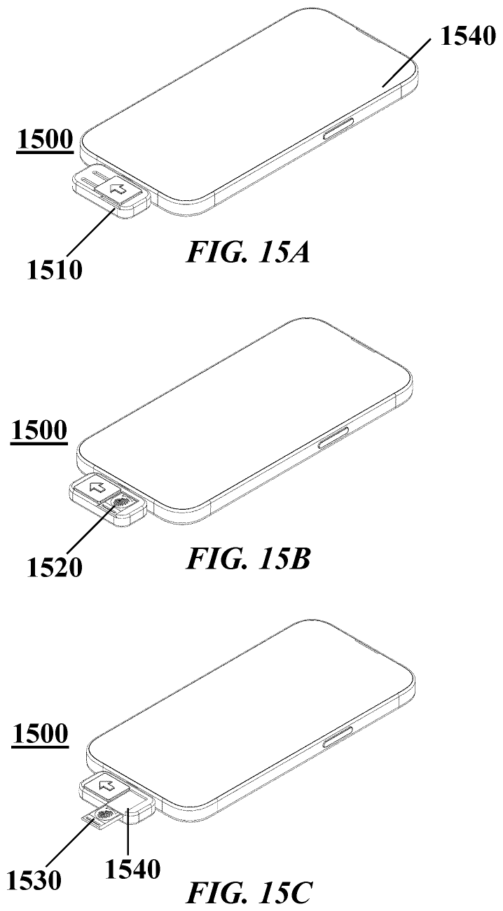

FIGS. 15A-15C show examples of a universal serial bus (USB) plug in form factor test strip reading device 1500. The USB plug in form factor test strip reading device may be configured to plug into a USB plug of, for example, a mobile phone 1540. The USB plug in form factor test strip reading device may comprise a lid 1510 configured to cover the test strip 1520, which may be removable 1530 from slot 1540. The USB port may provide data access as well as power delivery to the USB plug in form factor test strip reading device.

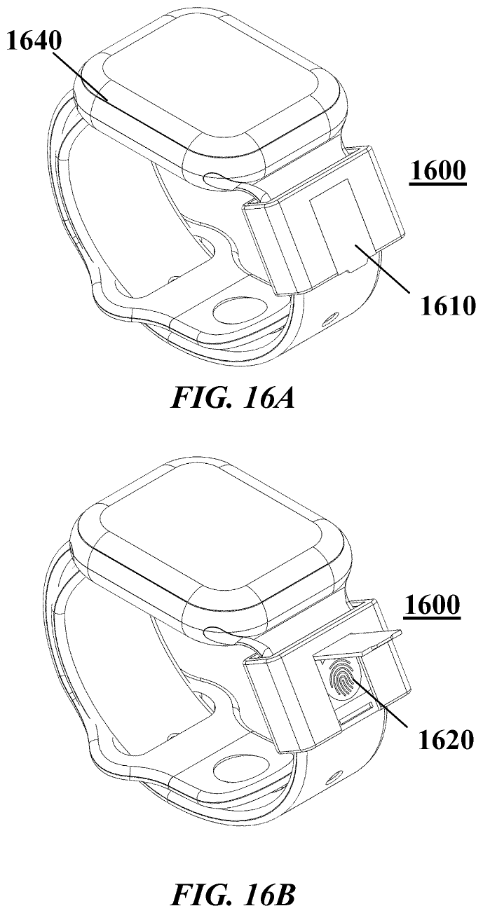

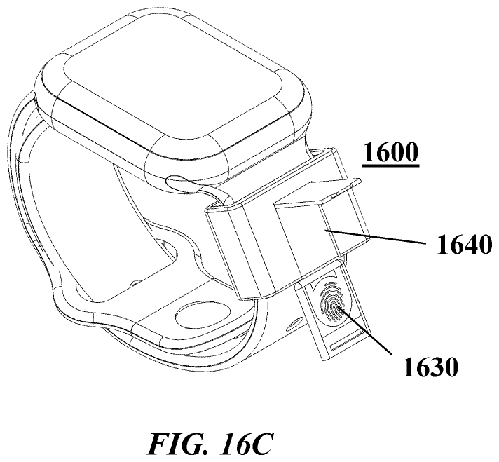

FIGS. 16A-16C show examples of a smart watch band form factor test strip reading device 1600. The smart watch 1640 may have the smart watch band form factor test strip reading device 1600 inserted through a band of the smart watch, thereby reversibly attaching the smart watch band form factor test strip reading device to the smartwatch. The smart watch band form factor test strip reading device may comprise a lid 1610 movable to expose the test strip 1620. The test strip may be removable 1630 from slot 1640. The smart watch band form factor test strip reading device may be configured to wirelessly communicate with the smartwatch. In some cases, the smart watch band form factor test strip reading device can be used with a non-smart watch.

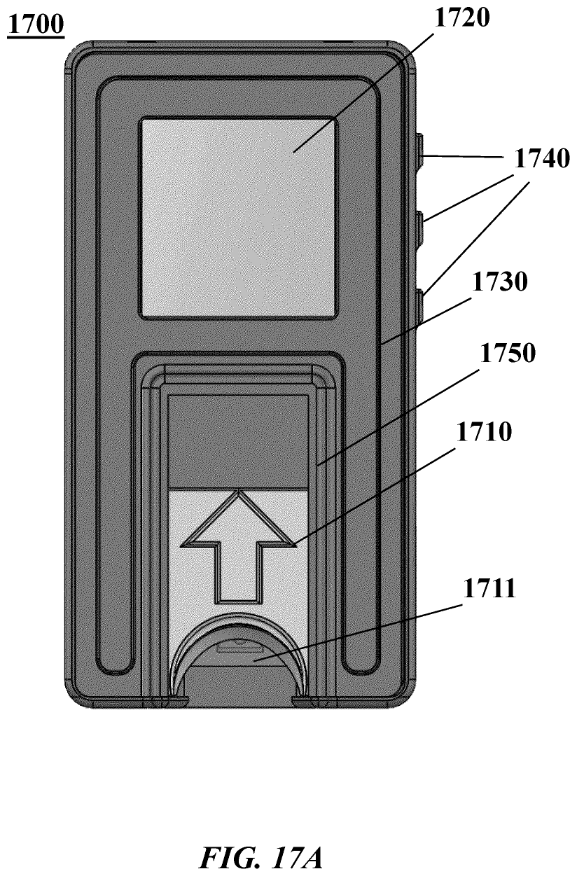

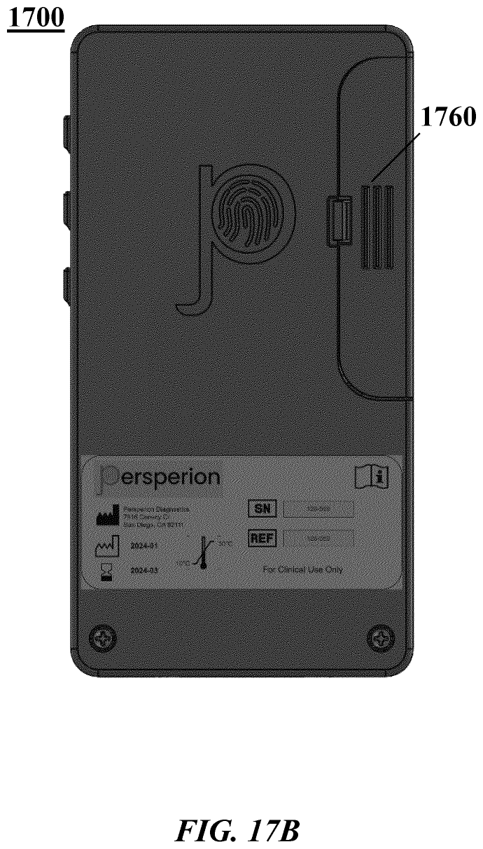

FIGS. 17A-17B show examples of a handheld form factor test strip reading device 1700. The handheld form factor test strip reading device can comprise a lid 1710 as described elsewhere herein. The lid may be slidable to the expose a test strip. The lid may comprise a void 1711 configured to have a user's finger interface with the lid to move the lid. The handheld form factor test strip reading device may comprise a ridge 1750 configured to guide a finger of the user to the lid 1710. The body 1730 of the handheld form factor test strip reading device can be configured to contain, for example, a battery, a battery cover 1760, a microprocessor, a screen 1720, a plurality of buttons 1740, or the like, or any combination thereof. The handheld form factor test strip reading device can be configured to implement the methods of the present disclosure (e.g., determine a blood glucose level of a user).

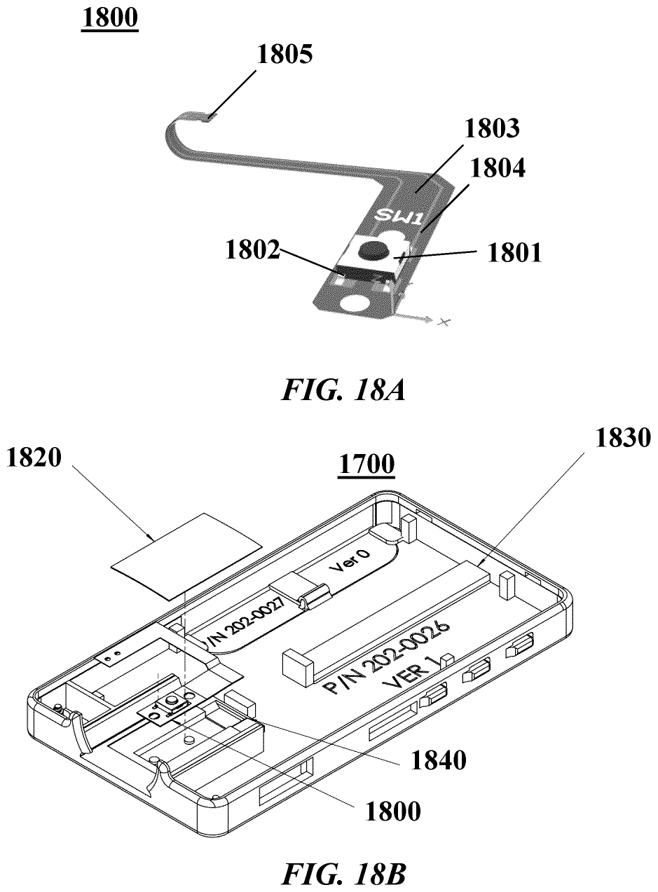

FIGS. 18A-18B show examples of a pressure switch 1800 and an integration into a test strip reading device 1700. The test strip reading device may be as described elsewhere herein. The test strip reading device may comprise a pressure switch 1800 configured to provide a reading about the presence and/or amount of pressure being applied to a test strip held in the test strip reading device. For example, the pressure switch can detect a pressure being applied to the test strip and begin, for example, a method of the present disclosure to determine a property of a biomarker in the sweat of a user. In another example, the pressure switch can record an amount of pressure being applied to the test strip throughout a reading, thereby providing feedback to the user about the amount of pressure being applied. In this example, if the user is providing too much or too little pressure, the test strip reading device can communicate (e.g., via a display) to the user to correct the amount of pressure being applied.

The pressure switch 1800 may comprise a switch unit 1801. The switch unit may be configured to receive movement from the test strip when, for example, a user places their finger on the test strip. The switch unit may comprise, for example, one or more electromechanical pressure switches, electronic pressure switches, solid-state pressure switches, capacitive pressure switches, or the like, or any combination thereof. In some cases, the switch unit is a tactile switch, an optical switch, or a combination thereof. The switch unit may comprise contacts 1802 configured to be in electrical communication with electrical leads 1804 that are a part of ribbon 1803. The ribbon and corresponding leads can enable connection of the switch unit in a low cost and facile to manufacture way. The ribbon may comprise end leads 1805, which can be used to connect the ribbon to the rest of the test strip reading device. The test strip reading device 1700 may comprise a bottom case 1830, a step block 1840 configured to protect the ribbon 1803 and leads 1804. An optional protective barrier 1820 may be positioned between the test strip and the switch unit to for example, prevent ingress of debris into the test strip reading device as well as protect the switch unit from wear. The protective barrier may comprise, for example, polyethylene terephthalate, polypropylene, polyvinyl chloride, polycarbonate, polyimide, polytetrafluoroethylene, or the like, or any combination thereof. The protective barrier may be configured to provide spring action configured to reset the test strip and the switch unit after the user has removed their finger. For example, the protective barrier may be raised in the middle over where the switch unit is. The raised portion can provide a spring force.

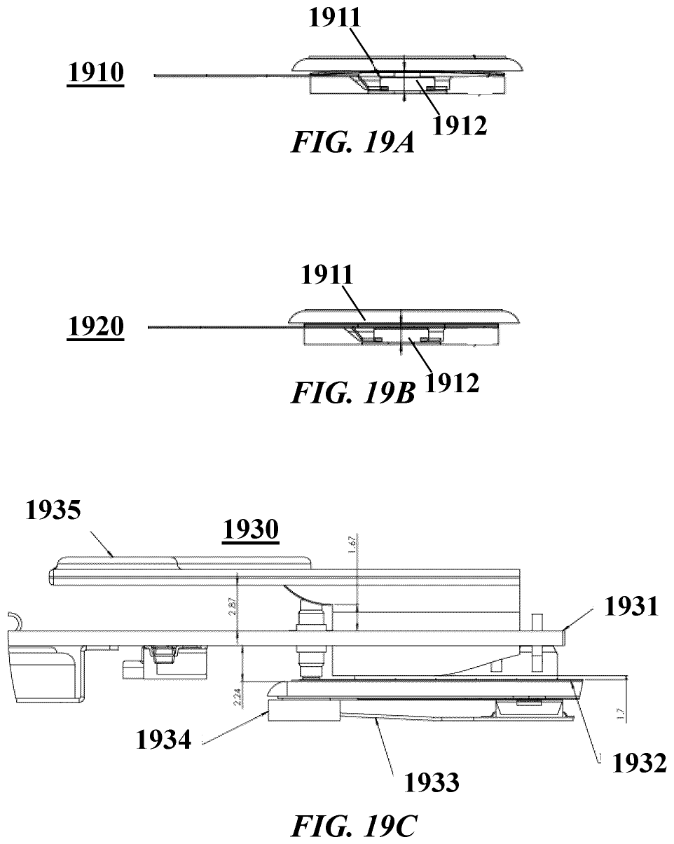

FIGS. 19A-19C show examples of a pressure switch in activation and in a test strip reading device. FIG. 19A shows a test strip 1911 positioned over a switch unit 1912, where the switch unit is not activated and the test strip does not have pressure 1910. In FIG. 19B, the test strip is under pressure 1920, and the switch unit is depressed and activated.

FIG. 19C shows an area 1930 around the test strip and the pressure switch, according to some embodiments. The printed circuit board 1931 may house processors and other electronic elements as described elsewhere herein. The test strip holder 1932 can be positioned above switch unit 1933 such that a test strip can be in movable communication with the switch unit. Step block 1934 can be positioned as a stop to prevent overtravel of the test strip as well as provide structural support for the test strip reading device. Lid 1935 may be as described elsewhere herein (e.g., configured to protect a test strip).

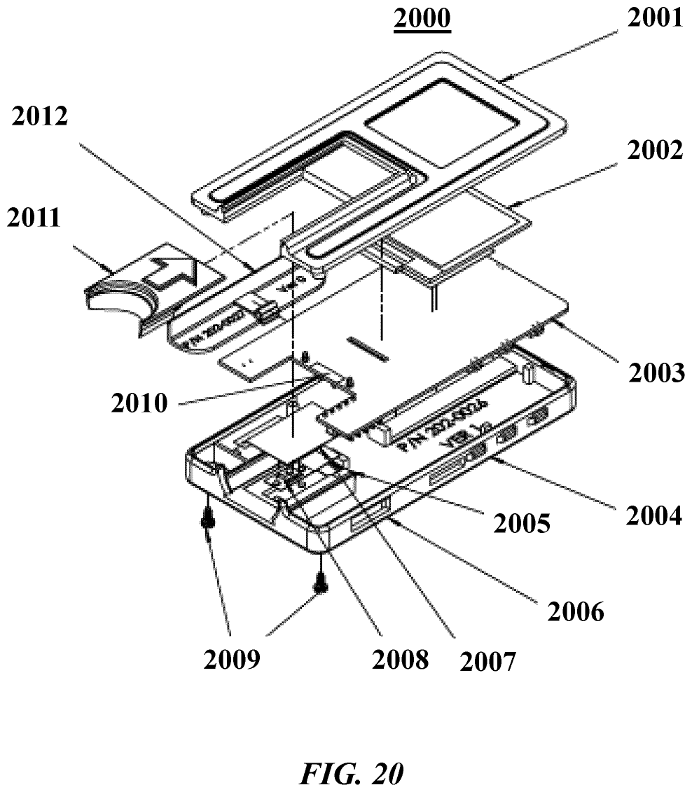

FIG. 20 shows an example of an exploded view of a test strip reading device 2000. The test strip reading device may comprise a top cover 2001 and a bottom cover 2004 configured to hold the elements of the test strip reading device. A display 2002 can be configured to provide information to the user of the test strip reading device. Printed circuit board 2003 can be configured to hold and interconnect electronic elements of the test strip reading device. The step block 2005 and protective barrier 2007 may be as described elsewhere herein. The switch unit 2008 can be configured as described elsewhere herein. Label 2006 can provide, for example, model information, a serial number, other listings, patent information, or the like, or any combination thereof. Screws 2009 can secure the top and bottom covers and hold the test strip reading device together. Insulating tape 2010 may be configured to provide insulation and may comprise, for example, Kapton tape. Lid 2011 may be configured to cover the test strip reading area and may be as described elsewhere herein. Battery cover 2012 may be configured to permit access to replaceable batteries as described elsewhere herein.

### Additional Numbered Embodiments

1. A portable, sweat-based analyte monitoring system, the system comprising:
a) a test strip comprising a solid-state electrochemical sensor layer and a conductive layer; and
b) a test strip reading device,
wherein the test strip is configured to contact a portion of skin of a user to receive a sample from the skin, and
wherein the test strip reading device is configured to receive the test strip.

2. The system of embodiment 1, wherein the sample comprises sweat.

3. The system of any one of preceding embodiments, wherein the conductive layer comprises a current conducting material.

4. The system of any one of preceding embodiments, wherein the current conducting material comprises one or more of a metal, a doped ceramic, a carbonaceous material, or a conductive polymer.

5. The system of embodiment 4, wherein the metal comprises one or more of gold, silver, copper, ruthenium, rhodium, platinum, bismuth, tungsten, iron, titanium, rhenium, osmium, or iridium.

6. The system of embodiment 4, wherein the doped ceramic comprises one or more of indium tin oxide, aluminum-doped zinc oxide, or fluorine-doped tin oxide.

7. The system of embodiment 4, wherein the carbonaceous material comprise one or more of graphite, carbon black, carbon nanotubes, graphene, or reduced graphene oxide.

8. The system of embodiment 4, wherein the conductive polymer comprises one or more of poly(3,4-ethylenedioxythiophene) polystyrene sulfonate, polypyrrole, polyaniline, poly phenylenediamine. polythiophene, or poly(p-phenylene).

9. The system of any one of preceding embodiments, wherein the conductive layer is configured to conduct an electrochemical signal and establish an electrical connection to the test strip reading device when the test strip is inserted into the test strip reading device.

10. The system of any one of preceding embodiments, wherein the sensor comprises an electrochemical transducer a biorecognition functionality.

11. The system of any one of preceding embodiments, wherein the sensor comprises one or more of an enzyme, an ionophore, a binding polypeptide, a polynucleotide, an aptamer, a molecularly imprinted polymer, or a microbe.

12. The system of embodiment 11, wherein the enzyme comprises one or more of glucose oxidase, glucose dehydrogenase, alcohol oxidase, alcohol dehydrogenase, lactate oxidase, lactate dehydrogenase, cholesterol oxidase uricase, urease, ascorbate oxidase, horseradish peroxidase, catalase, tyrosinase, creatinine deiminase, amylase, glutamate oxidase, xanthine oxidase, bilirubin oxidase, hydroxybutyrate dehydrogenase, hydroxybutyrate oxidase, D-amino acid oxidase, L-amino acid oxidase, pyruvate oxidase, or acetoacetate dehydrogenase.

13. The system of embodiment 11, wherein the binding polypeptide comprises an antibody or an antigen binding fragment.

14. The system of any one of preceding embodiments, wherein the sensor further comprises one or more of a cofactor, a mediator, a stabilizer, a surfactant, a polymer, a plasticizer, a cross-linker, cross-linking terminator, or a cross-linking initiator.

15. The system of embodiment 14, wherein the cofactor comprises one or more of nicotinamide adenine dinucleotide, nicotinamide adenine dinucleotide phosphate, flavin adenine dinucleotide, flavin mononucleotide, thiamine pyrophosphate, biotin, heme, coenzyme A, coenzyme Q, cobalamin, pyridoxal phosphate, tetrahydrofolic acid, or S-adenosyl methionine.

16. The system of embodiment 14, wherein the mediator comprises one or more of ferrocene and ferrocene derivatives, ferricyanide, ferrocyanide, Prussian blue, quinones, tetrathiafulvalene, organic dyes (methylene blue, Meldola blue), osmium complex, or ruthenium complex.

17. The system of embodiment 14, wherein the stabilizer comprises one or more of glycerol, trehalose, chitosan, albumin, polyethylene glycol, calcium chloride, silica, polyols, diethyldithiocarbamate, or mercaptoundecanol.

18. The system of embodiment 14, wherein the surfactant comprises a Pluronic triblock copolymer, fluorosurfactant, tetramethyldecynediol, triton-X 100, trion-X 114.

19. The system of embodiment 14, wherein the polymer comprises one or more of alginate, chitosan, acrylate, methacrylate, or saccharides.

20. The system of embodiment 14, wherein the polymer comprises a monomer of the polymers.

21. The system of embodiment 14, wherein the plasticizer comprises one or more of di-2-ethylhexyl phthalate, diisononyl phthalate, dioctyl adipate, diisononyl adipate, triphenyl phosphate, polyethylene glycol, dibutyl sebacate, dioctyl sebacate, acetyl tributyl citrate, or epoxidized soybean oil.

22. The system of embodiment 14, wherein the cross-linker comprises one or more of glutaraldehyde, 1-ethyl-3-(3-dimethylaminopropyl)carbodiimide, N-hydroxysuccinimide, polyethylene glycol diglycidyl ether, glyoxal, benzophenone, disuccinimidyl suberate, bis(sulfosuccinimidyl) suberate, or dithiobis(succinimidyl propionate).

23. The system of embodiment 14, wherein the cross-linking initiator comprises one or more of azobisisobutyronitrile, potassium persulfate, ammonium persulfate, tetramethylethylenediamine, benzoyl peroxide, or Irgacure 2959.

24. The system of any one of preceding embodiments, wherein the test strip further comprises a substrate.

25. The system of any one of preceding embodiments, wherein the substrate comprises one or more of plastics, ceramics, or natural materials.

26. The system of any one of preceding embodiments, wherein the substrate comprises one or more of polyethylene, polypropylene, polyethylene terephthalate, polyester, polyimide, polydimethylsiloxane, alumina, silicon, or paper.

27. The system of any one of preceding embodiments, wherein the substrate is treated mechanically, chemically, and/or physically to change a characteristic.

28. The system of any one of preceding embodiments, wherein the characteristic comprises one or more of surface roughness, surface cleanliness, hydrophobicity, dielectric constant, or electrostatic charge.

29. The system of any one of preceding embodiments, wherein the substrate treatment comprises one or more of base chemical etching, plasma etching, electroplating, abrasion, laser ablation, sputtering, heating, and physical or chemical vapor deposition.

30. The system of any one of preceding embodiments, wherein the substrate treatment improves adhesion of the sample and/or compatibility of the sample.

31. The system of any one of preceding embodiments, wherein the test strip further comprises an insulation layer.

32. The system of any one of preceding embodiments, wherein the insulation layer comprises a non-conductive material.

33. The system of any one of preceding embodiments, wherein the insulation layer is waterproof.

34. The system of any one of preceding embodiments, wherein the insulation layer is rigid.

35. The system of any one of preceding embodiments, wherein the insulation layer is transparent.

36. The system of any one of preceding embodiments, wherein the insulation layer has an antibacterial property.

37. The system of any one of preceding embodiments, wherein the insulation layer covers a portion of the test strip to define an exposed area for contacting the skin.

38. The system of any one of preceding embodiments, wherein the insulation layer is deposited or attached to another layer of the test strip.

39. The system of any one of preceding embodiments, wherein the test strip further comprises a counter-interference layer.

40. The system of any one of preceding embodiments, wherein the counter-interference layer comprises one or more of a metal, a mediator, a negatively or positively charged molecule.

41. The system of embodiment 40, wherein the mediator comprises one or more of ferrocene and ferrocene derivatives, ferricyanide, ferrocyanide, Prussian blue, quinones, tetrathiafulvalene, an organic dye, an osmium complex, or a ruthenium complex.

42. The system of embodiment 41, wherein the organic dye comprises one or more of methylene blue or Meldola blue.

43. The system of embodiment 40, wherein the negatively or positively charged molecule comprises one or more of Nafion, polyfluoroalkanes, polychlorotrifluoroethylene, fluorine rubber, chitosan, polyethyleneimine, polyurethane, polystyrene sulfonate, polyvinyl sulfate, polyvinyl alcohol, or polyvinyl chloride.

44. The system of embodiment 43, wherein the fluorine rubber comprises one of more of copolymer of vinylidene fluoride, tetrafluoroethylene, hexafluoropropylene, and perfluoromethylvinylether.

45. The system of embodiment 43, wherein the fluorine rubber comprises the addition of ethylene and propylene in the copolymer.

46. The system of any one of preceding embodiments, wherein the test strip further comprises a protective layer.

47. The system of any one of preceding embodiments, wherein the protective layer comprises a polymer.

48. The system of embodiment 45, wherein the polymer comprises one or more of Nafion, chitosan, methyl- or ethyl-cellulose, polyvinyl chloride, polyurethane, silicone, polytetrafluoroethylene, polyolefin, polyester, polycarbonate, copolymers, polyvinylidene fluoride, polymethyl methacrylate, polyvinyl alcohol, polyethylene glycol, poly acrylamide, polyacetate, polyvinylpyrrolidone, polyethylene oxide, poly(p-phenylenediamine), or polysulfides.

49. The system of any one of preceding embodiments, wherein the protective layer is permeable.

50. The system of any one of preceding embodiments, wherein the protective layer is configured to protect the layers beneath from mechanical and/or chemical damage.

51. The system of any one of preceding embodiments, wherein the test strip further comprises a conditioning layer.

52. The system of any one of preceding embodiments, wherein the conditioning layer comprises a polymer and a surfactant.

53. The system of any one of preceding embodiments, wherein the conditioning layer is configured to adjust hydrophilicity and electrostatic charge of the electrode.

54. The system of any one of preceding embodiments, wherein the test strip is single-use.

55. The system of any one of preceding embodiments, wherein the test strip is reusable for at least 10 uses.

56. The system of any one of preceding embodiments, wherein a layer is patterned for improved sweat contact and signal transduction.

57. The system of embodiment 54, wherein the pattern comprises one or more of an interdigitated pattern, a concentric pattern, a radially interdigitated pattern.

58. The system of embodiment 54 or 55, wherein each segment of a line or an arc of the pattern is about 1 mm away from each other

59. The system of embodiment 54, 55, or 56, wherein a width of the line or the arc of the pattern is no more than about 1 mm.

60. The system of any one of preceding embodiments, wherein the analyte comprises one or more of sodium, potassium, calcium, magnesium, chlorides, fluorides, glucose, lactates, alcohols (ethanol), ketones (β-hydroxybutyrate, acetoacetate), cortisol, uric acid, urea, ascorbates, creatinine, creatine, amino acids (glycine, leucine, proline, lysine, alanine, glutamine, tyrosine, tryptophan, cysteine leucine, proline, lysine, alanine, glutamine, tyrosine, tryptophan, cysteine), levodopa, caffeine, cannabinoids, cocaine, opioids, explosives, or nerve agents.

61. The system of any one of preceding embodiments, wherein the device comprises a housing and a circuit board.

62. The system of any one of preceding embodiments, wherein the circuit board comprises a microcontroller, a signal generation and processing chip, a data storage chip, and a connector configured to receive the test strip.

63. The system of any one of preceding embodiments, wherein the circuit board further comprises one or more of a wireless transmission functionality, an antenna, a display panel, an indicator light, a user input button, or a power source.

64. The system of embodiment 61, wherein the user input button comprises an electrical button or a mechanical button or a combination thereof.

65. The system of any one of preceding embodiments, wherein the circuit board is configured to generate an electrochemical measurement when the test strip is inserted into the device and the sample is contacted onto the test strip.

66. The system of any one of preceding embodiments, wherein the electrochemical measurement comprises one or more of open circuit voltage measurement, chronopotentiometry, chronoamperometry, chronocoulometry, cyclic voltammetry, differential pulse voltammetry, square wave voltammetry, linear sweep voltammetry, alternating current impedance spectrometry, or direct current internal resistance measurement.

67. The system of any one of preceding embodiments, wherein the circuit board is configured to generate an electrical signal from an electrochemical measurement from the sample and process the electrical signal to determine a concentration of the analyte.

68. The system of any one of preceding embodiments, wherein the circuit board processes the electrical signal into the concentration of the analyte using a pre-programmed algorithm and a user-input calibration data.

69. The system of any one of preceding embodiments, wherein the pre-programmed algorithm comprises a two-point or single-point linear regression using the user-input calibration data.

70. The system of any one of preceding embodiments, wherein the user-input calibration data is obtained from another analyte monitoring device.

71. The system of any one of preceding embodiments, wherein the another analyte monitoring device comprises a blood glucose monitor or a continuous glucose monitor.

72. The system of any one of preceding embodiments, wherein the pre-programmed algorithm is personalized to the user.

73. The system of any one of preceding embodiments, wherein the pre-programmed algorithm is individualized for different fingers of the user.

74. The system of any one of preceding embodiments, wherein the data storage chip is configured to store one or more of a number of uses of the test strip, a raw signal, processed data, time of measurement, or the personalized and individualized calibration data.

75. The system of any one of preceding embodiments, wherein the circuit board is configured to deliver the stored information to a secondary device.

76. The system of any one of preceding embodiments, wherein the secondary device comprises one or more of a computer, a mobile device, or a cloud-based data center.

77. The system of any one of preceding embodiments, wherein the secondary device performs data analysis and data visualization on the stored information.

78. The system of any one of preceding embodiments, wherein the device further comprises one or more of an optical sensor or an electrical sensor.

79. The system of any one of preceding embodiments, wherein the optical sensor or the electrical sensor is configured to measure one or more physiological and physical signals from the skin.

80. The system of any one of preceding embodiments, wherein the other one or more physiological and physical signals comprises one or more of temperature, moisture level, sweat gland density, sweat rate, transdermal water loss rate, pressure, hydration level, heart rate, or blood oxygen level.

81. The system of any one of preceding embodiments, wherein the pre-programmed algorithm uses the other one or more physiological and physical signals to make a correction in the calculation of the analyte concentration from the electrical signal.

82. The system of any one of preceding embodiments, wherein the device further comprises an optical, a capacitive, or an ultrasonic fingerprint scanner to identify the user and the finger contacting the test strip.

83. The system of any one of above embodiments, wherein the housing is configured to protect the circuit board and the test strip when inserted into the device.

84. The system of any one of above embodiments, wherein the housing comprises a moving cover to cover a portion of the test strip inserted into the device when the moving cover is closed and to uncover the portion of the test strip when the moving cover is opened.

85. The system of any one of above embodiments, wherein the cover comprises a replaceable wipe or a squeegee to remove the sample residue after contact with the skin.

86. The system of any one of above embodiments, wherein the housing comprises a replaceable wiper for wiping the skin prior to contacting the test strip.

87. The system of any one of above embodiments, wherein the housing comprises a mechanical guide for contact by the skin.

88. A method of non-invasively detecting an analyte from a fingertip, the method comprising:
a) inserting the test strip of any one of preceding embodiments into the device of any one of preceding embodiments;
b) placing a portion of skin of a user in contact with the test strip; and
c) obtaining a reading of the analyte from the test strip reading device.

89. A method of non-invasively monitoring an analyte from a fingertip, the method comprising:
a) receiving a sample from a skin of a user on the test strip of any one of preceding embodiments inserted into the device of any one of preceding embodiments by contact of the skin to the test strip;
b) generating an electrical signal from an electrochemical measurement of the analyte using the solid-state electrochemical sensor;
c) measuring a concentration of the analyte in the sample from the electrical signal;
d) calculating, by the device, a systemic concentration of the analyte in the user from the concentration of the analyte in the sample; and
e) providing the systemic concentration of the analyte on a display of the device.

90. A method of preparing a test strip for a sweat-based analyte monitoring system, the method comprising:
a) deposition of one or more layers on top of a substrate; and
b) attaching the deposited layer to the substrate or the previous layer,
   wherein the test strip comprises the substrate, a conductive layer, a solid-state electrochemical sensor, and an insulation layer.

91. The method of embodiment 88, wherein the deposition comprises one or more of electrodeposition, sputtering, physical and chemical vapor deposition, screen printing, transfer printing, dip coating, spray coating, spin coating, doctor blading, inkjet printing, drop casting, or microdot patterning.

92. The method of embodiment 88 or 89, wherein attaching the layer comprises one or more of cold pressing, hot pressing, adhesion, crimping, or embedding.

93. The method of any one of embodiments 88 to 90, wherein a layer is patterned to improve sweat contact and signal transduction.

94. The method of any one of embodiments 88 to 91, wherein patterning comprises one or more of direct deposition or printing, masked deposition or printing, masked etching, laser ablation, laser cutting, mechanical abrasion, or mechanical cutting.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A portable glucose monitoring system, the system comprising:
a test strip comprising a plurality of first electrodes and a plurality of second electrodes, wherein the plurality of first electrodes and the plurality of second electrodes are alternately arranged; and
a test strip reading device comprising a slot configured to receive a first portion of the test strip and to keep a second portion of the test strip accessible to a contact by a skin of a user,
wherein a second portion of the test strip is configured to receive a sweat sample from the skin of the user by the contact of the skin to the second portion, and wherein the test strip is configured to detect glucose in the sweat sample.

2. The system of claim 1, wherein the test strip reading device comprises a lid configured to cover the second portion of the test strip when the test strip is placed into the slot.

3. The system of claim 2, wherein the lid is movable to uncover the second portion of the test strip when the test strip is placed into the slot.

4. The system of claim 2, wherein the test strip reading device comprises a lid sensor configured to detect an open state or a closed state of the lid.

5. The system of claim 4, wherein the lid sensor comprises a pressure sensor, a magnetic sensor, a positional sensor, a light sensor, an electrical sensor, or any combination thereof.

6. The system of claim 1, wherein the test strip reading device comprises a pressure switch positioned below the slot.

7. The system of claim 6, wherein the pressure switch is configured to be below the second portion of the test strip when the test strip is placed into the slot.

8. The system of claim 6, wherein the pressure switch is configured to determine at least one of a presence or a pressure of the contact by the skin of the user.

9. The system of claim 6, wherein the pressure switch comprises an electromechanical pressure switch, an electronic pressure switch, a solid-state pressure switch, a capacitive pressure switch, or any combination thereof.

10. The system of claim 6, wherein the system comprises a protective barrier positioned between the pressure switch and the test strip.

11. The system of claim 10, wherein the protective barrier is configured to reset the test strip upon termination of the contact by the skin of the user.

12. The system of claim 1, wherein the test strip reading device comprises a haptic feedback module configured to provide a vibrational feedback when the user activates or deactivates the test strip reading device.

13. The system of claim 1, wherein the system comprises a computer processor operably coupled to the test strip reading device and configured to determine a glucose concentration in the sample.

14. The system of claim 1, wherein the system comprises a wireless communication module, wherein the wireless communication module is configured to communicate with a computing device, wherein the computing device comprises a mobile phone, a watch, a wearable device, a laptop computer, or any combination thereof.

15. The system of claim 1, wherein the system comprises a sensor configured to measure one or more of a signal of the user, wherein the signal comprises temperature, a moisture level, a sweat gland density, a sweat rate, a transdermal water loss rate, a pressure, a hydration level, a heart rate, or a blood oxygen level.

16. The system of claim 1, wherein the test strip reading device is calibrated by a concentration of glucose in a blood sample of the user.

17. The system of claim 1, wherein the test strip comprises an electrochemical sensor layer comprising glucose oxidase disposed upon at least one of the plurality of first and second electrodes.

18. The system of claim 1, wherein the test strip comprises a second layer, wherein the second layer comprises one or more of alginate, chitosan, acrylate, methacrylate, saccharides, or polyethyleneimine (PEI).

19. The system of claim 1, wherein the test strip comprises a third layer, wherein the third layer comprises one or more of Nafion, polychlorotrifluoroethylene, polyfluoroalkanes, fluorine rubber, chitosan, polyethyleneimine, polyurethane, poly(p-phenylenediamine), polystyrene sulfonate, polyvinyl sulfate, polyvinyl alcohol, or polyvinyl chloride.

20. A method of determining a blood glucose concentration of the user, the method comprising:
a) providing the portable glucose monitoring system of claim 1;
b) contacting a portion of the skin of the user to the second portion of the test strip;
c) detecting an electrochemical signal using the plurality of first and second electrodes; and
d) determining the blood glucose concentration of the user with the test strip reading device based at least on the electrochemical signal.
